(19) United States
(12) Patent Application Publication
(10) Pub. No.: US 2024/0293071 A1
(43) Pub. Date: Sep. 5, 2024

(54) SYSTEM, METHOD AND APPARATUS FOR REAL-TIME 3D CARDIAC MAPPING WITH MULTI-CATHETER SUPPORT AND CARDIAC-WALL ANALYTICS

(71) Applicant: Garth Constantine, Murrieta, CA (US)

(72) Inventors: Garth Constantine, Murrieta, CA (US); Noam Seker-Gafni, Irvine, CA (US)

(21) Appl. No.: 18/592,453

(22) Filed: Feb. 29, 2024

Related U.S. Application Data
(60) Provisional application No. 63/576,784, filed on Mar. 3, 2023.

(51) Int. Cl. A61B 5/367; A61B 5/06; A61B 5/287; A61B 5/339; A61B 8/12; A61B 34/20

(52) U.S. Cl. CPC A61B 5/367; A61B 5/062; A61B 5/066; A61B 5/068; A61B 5/287; A61B 5/339; A61B 34/20; A61B 8/12; A61B 2034/2061

(57) ABSTRACT

A system for surgical imaging is disclosed. The system utilizes imaging devices configured to be inserted into a body of a patient. The system includes a display device, a first catheter having a first transducer. The first transducer is configured to sense an anatomical structure of the body and output a first set of imaging data. The system includes a second catheter having a second transducer configured to sense the anatomical structure and output a second set of imaging data. The system includes a processor in communication with a memory storing executable instructions. The system receives, simultaneously, the first set and second set of imaging data. The system determines positions of the anatomical structures, based on the first and second set of imaging data. The system displays, on the display device, a real-time rendering of the anatomical structure based on the position of the anatomical structure.

### CROSS-REFERENCE TO RELATED APPLICATIONS

This application claims priority under 35 U.S.C. § 119(e) from provisional U.S. patent application No. 63/576,784, filed Mar. 3, 2023, the contents of which are incorporated herein by reference.

### BACKGROUND

The field of interventional cardiology and electrophysiology has evolved with the use of various systems and tools to support minimally invasive cardiovascular procedures. These advancements address the inherent challenge of operating without direct visual access to catheters and devices used in diagnosing and treating cardiac diseases and congenital conditions. Complete 3D reconstructions of cardiac anatomy by static imaging solutions like rotational angiography (Angio) or Magnetic Resonance Imaging (MRI) is known. The static nature of such methods limits real-time applications during catheterization.

For example, fluoroscopy, commonly used in cardiovascular procedures, provides 2D real-time imaging of non-radiolucent objects and cardiac tissue. Despite being a standard of care, fluoroscopy relies on ionizing radiation, necessitating protective measures for physicians and lab staff, such as wearing heavy lead aprons. This not only limits mobility but also falls short in clearly imaging critical anatomical structures like valve leaflets.

In electrophysiology, 3D Electrophysiology (EP) mapping systems are employed. Such systems integrate intra-cardiac Electrogram (EGM) signal sensing from electrodes on catheters, localizing these electrodes in a 3D space to model cardiac chamber walls. EGM-derived data is then projected onto these models. While widely used for diagnosing arrhythmia mechanisms and guiding ablation therapy, such reconstructions can be partial and inaccurate, potentially leading to suboptimal diagnosis and misplaced ablation targets. Transesophageal Echocardiogram (TEE) is another imaging modality used in catheterization procedures, which involves placing an ultrasound transducer in the esophagus to visualize cardiac anatomy. This method requires full anesthesia due to patient discomfort and has limitations when the imaging source is positioned in the esophagus.

Intra-cardiac Echocardiography (ICE) utilizing an ultrasound transducer integrated into a catheter is a widely used imaging modality. ICE transducer catheters provides valuable information about anatomical structures and device positioning. However, current implementations of ICE catheters limit the use to a single ICE catheter per procedure. Frequently, the ICE catheter must be precisely angled or repositioned for optimal imaging and modeling of cardiac chambers, necessitating extensive manual adjustments for accurate visualization.

### SUMMARY

Accordingly, some embodiments include a system for surgical imaging utilizing imaging devices configured to be inserted into a body of a patient. In some embodiments, the system includes a display device, a first catheter having a first transducer, the first transducer configured to sense an anatomical structure of the body of the patient and output a first set of imaging data. In some embodiments, the system includes a second catheter having a second transducer configured to sense the anatomical structure and output a second set of imaging data, a processor in communication with a memory. The memory stores executable instructions that when executed by the processor configure the system for receiving, simultaneously, the first set of imaging data and the second set of imaging data and determining, a position of the anatomical structure, based on the first set of imaging data and the second set of imaging data. In some embodiments, the system is configured for displaying, on the display device, a real-time rendering of the anatomical structure based on the position of the anatomical structure.

Some embodiments include an apparatus configured to be surgically positioned into a body of a patient. In some embodiments, the apparatus includes a catheter shaft coupled to a handle. In some embodiments, a plurality of electrodes are positioned on the catheter shaft and, in some embodiments, a magnetic sensor is positioned on the catheter shaft. In some embodiments, the apparatus includes a transducer configured to sense one or more anatomical structures of the body of the patient and output a first set of imaging data. In some embodiments, the magnetic sensor tracks a location and position of the plurality of electrodes and/or the transducer.

Some embodiments include a method for surgical imaging of anatomical structures of a body of a patient utilizing a system for surgical imaging including a display device and one or more catheters. The method includes establishing connections to a first transducer, a second transducer, and a plurality of electrodes and receiving, from the first transducer, a first data stream corresponding to an anatomical structure of the body of the patient. In some embodiments, the method includes receiving, from the second transducer, a second data stream corresponding to the anatomical structure and receiving, from the plurality of electrodes, voltage data corresponding to impedance values. In some embodiments, the method includes determining, based on the first data stream, the second data stream, and the impedance values, a position and orientation of the anatomical structure, the plurality of electrodes, and the first transducer, and/or the second transducer. In some embodiments, the method includes reconstructing, based on the position and orientation of the anatomical structure, a single real-time imaging stream of the anatomical structure, and transmitting the single-real time imaging stream for display on a display device.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is noted, however, that the appended drawings illustrate only some aspects of this disclosure and the disclosure may admit to other equally effective embodiments.

To facilitate understanding, identical reference numerals have been used, where possible, to designate identical elements that are common to the figures. It is contemplated that elements and features of one embodiment may be beneficially incorporated in other embodiments without further recitation.

FIG. 1A illustrates a schematic of an exemplary intra-cardia echocardiography (ICE) surgical system, in accordance with some embodiments;

FIGS. 1B-1C illustrate schematics of cardiac structures with an ICE catheter deployed, in accordance with some embodiments;

FIGS. 2A-2J illustrates schematics of an exemplary ICE catheters for use in the system shown in FIG. 1A, in accordance with some embodiments;

FIGS. 3A-3E illustrate schematics of an exemplary ICE catheters for use in the system shown in FIG. 1A, in accordance with some embodiments;

FIG. 4 illustrates a schematic of an exemplary ICE catheter imaging console, in accordance with some embodiments;

FIG. 5A-5C illustrate schematics of a graphical user interface of a display device for an ICE surgical system;

FIGS. 6A-6B illustrate schematics of a coordinate system, in accordance with some embodiments;

FIGS. 7A-7E illustrate schematics of a graphical user interface for a display device of an ICE surgical system, in accordance with some embodiments; and

FIG. 8 depicts a flow chart corresponding to processes for ICE catheter deployment, in accordance with some embodiments.

### DETAILED DESCRIPTION

The present disclosure will now be described in detail with reference to the drawings, which are provided as illustrative examples of the disclosure so as to enable those skilled in the art to practice the disclosure. Notably, the figures and examples below are not meant to limit the scope of the present disclosure to a single embodiment, but other embodiments are possible by way of interchange of some or all of the described or illustrated elements. Moreover, where certain elements of the present disclosure can be partially or fully implemented using known components, only those portions of such known components that are necessary for an understanding of the present disclosure will be described, and detailed descriptions of other portions of such known components will be omitted so as not to obscure the disclosure.

As used herein, the singular form of “a”, “an”, and “the” include plural references unless the context clearly dictates otherwise. As used herein, the statement that two or more parts or components are “coupled” shall mean that the parts are joined or operate together either directly or indirectly (i.e., through one or more intermediate parts or components, so long as a link occurs). As used herein, “directly coupled” means that two elements are directly in contact with each other. As used herein, “fixedly coupled” or “fixed” means that two components are coupled so as to move as one while maintaining a constant orientation relative to each other. As used herein, “operatively coupled” means that two elements are coupled in such a way that the two elements function together. It is to be understood that two elements “operatively coupled” does not require a direct connection or a permanent connection between them. As utilized herein, “substantially” means that any difference is negligible, or that such differences are within an operating tolerance that are known to persons of ordinary skill in the art and provide for the desired performance and outcomes as described in one or more embodiments herein. Descriptions of numerical ranges are endpoints inclusive.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality). Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

Embodiments described as being implemented in hardware should not be limited thereto, but can include embodiments implemented in software, or combinations of software and hardware, and vice-versa, as will be apparent to those skilled in the art, unless otherwise specified herein. In the exemplary embodiments described herein, an embodiment showing a singular component should not be considered limiting; rather, the invention is intended to encompass other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present invention encompasses present and future known equivalents to the known components referred to herein by way of illustration.

The embodiments described herein relate generally to real time imaging surgical consoles and systems, components thereof, and methods of use thereof. For example, real-time 3D imaging is highly valuable in various types of cardiac treatments, particularly those requiring precise navigation and detailed visualization of the heart's anatomy and functions. Some of these treatments include, for example cryoablation, Radiofrequency Ablation (RFA), Pulsed Field Ablation (PFA), Transcatheter Aortic Valve Replacement (TAVR), Left Atrial Appendage Closure (LAAC), or Septal Defect Repair. In all the foregoing procedures, real-time 3D imaging enhances the physician's ability to perform complex interventions with increased precision and safety, reducing the risk of complications and improving patient outcomes. For clarity a brief review follows:

For example, cryoablation involves freezing the heart tissue to disrupt faulty electrical pathways are used to treat cardiac arrhythmias like atrial fibrillation. The system and apparatus described herein for real-time 3D imaging aids in accurately guiding the cryoablation catheter to the target areas within the heart. RFA, similar to cryoablation, yet using heat instead of cold, RFA is another technique for treating arrhythmias. PFA, using short electrical pulses to cause minimally-thermal irreversible electroporation, is another form of ablation widely used to ablate and treat cardiac arrhythmia. Such precise 3D imaging ensures the ablation catheter is correctly positioned to effectively treat the abnormal or targeted tissue without damaging surrounding areas.

In TAVR, for example, the real-time 3D imaging embodiments described here are advantageous for placing a replacement valve via a catheter, particularly in navigating the catheter and positioning the valve accurately within the aortic valve space. As another example, LAAC is a procedure that involves placing a device to close the left atrial appendage in patients with atrial fibrillation to reduce the risk of stroke. For example, one or more embodiments for real-time 3D imaging described herein are advantageous for precise placement of the closure device.

Septal Defect Repair (SDR) procedures also benefit from 3D imaging. SDR repairs atrial or ventricular septal defects (holes in the heart's walls). Advantageously, 3D imaging facilitate accurate placement of closure devices and assessment of cardiac function during procedures. For example, during electrophysiology studies and mapping, doctors may map the heart's electrical activity, for example, in cases of arrhythmia. Thus, real-time 3D imaging of the embodiments herein advantageously enhances the accuracy of such mapping and guides therapeutic interventions.

Accordingly, one or more embodiments herein include a system for providing accurate real time static and dynamic representation of the cardiac chambers' walls, valves, veins, ostia as well as other clinically relevant anatomical cardiovascular structures. In some embodiments, the system implements multiple intra-cardiac echocardiography (ICE) ultrasound imaging transducers embedded in intra-cardiac catheters, sheaths, or as a standalone catheter. In some embodiments, implanting multiple transducers in a single catheter advantageously provides real-time images with varying angles to the anatomy of interest.

Some embodiments disclosed herein may present real-time and static 2D and 3D images resulting from the one or more transducers (e.g., phase array transducers) and combine the 2D ultrasound image from individual so-called “slices” into a full visual reconstruction, or rendering, of the cardiac chambers resulting in a substantially complete shell of some, or all 4 chambers, which is discussed in further detail below. In some embodiments, the cardiac chambers' reconstruction may be displayed statically, for example, by freezing the shell to match any time across the cardiac cycle. In some embodiments, the cardiac chambers' reconstruction may be displayed dynamically, presenting wall motion and valve functions corresponding to the cardiac cycle in real time.

In some embodiments, the surgical system described herein may be capable of recording, analyzing and displaying electrical conductivity. Electrical conductivity data may facilitated by body surface ECG leads and/or intra-cardiac EGM associated with electrodes touching, or in close proximity to, the cardiac tissue. Such various cardiac electrical conductivity data (e.g., uni-polar voltage, bi-polar voltage, local activation time, low voltage signals, and/or fractionated signals), may be advantageously projected on the reconstruction. In some embodiments, such projection may be synchronized with the cardiac cycle to present the mechanical motion of the cardiac muscle with the corresponding cardiac electrical conduction mechanism. In some embodiments, in addition to cardiac electrical conductivity data, the system is capable of projecting and associating additional functional and/or visual indications on the rendered reconstruction as well as the 2D and 3D ultrasound images, which is described in detail below.

Some embodiments disclosed herein may advantageously be applied for diagnostic applications in electrophysiology, where accurate anatomy is advantageous to diagnose and treat cardiac arrhythmia, for example, with abnormal anatomy and hard-to-reach anatomical structures, as well as in left atrial appendage occlusion, structural heart procedures and congenital heart disease where the cardiac tissue and muscle mechanical characteristics are important for diagnosis and device placement. Overall, the systems, methods, and apparatus of the embodiments herein represent significant advancements in medical imaging technology, particularly in cardiac care, by providing enhanced visualization, accurate device localization, and sophisticated anatomical reconstruction capabilities, which are described in further detail below.

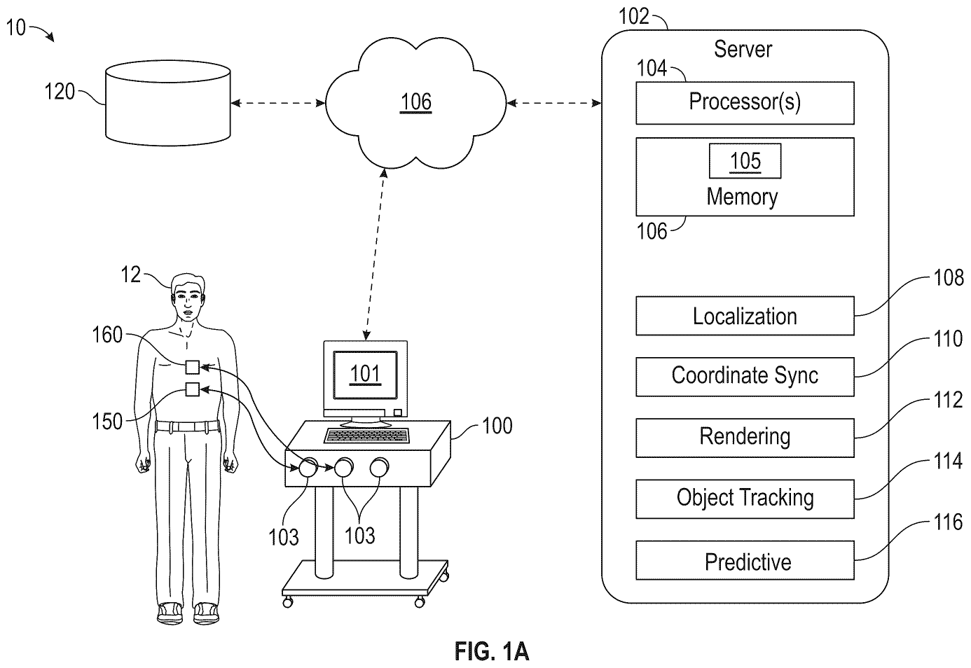

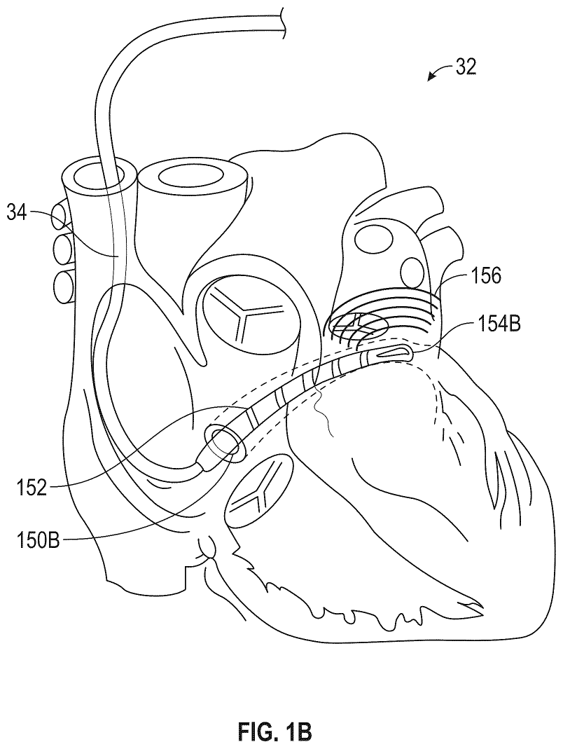

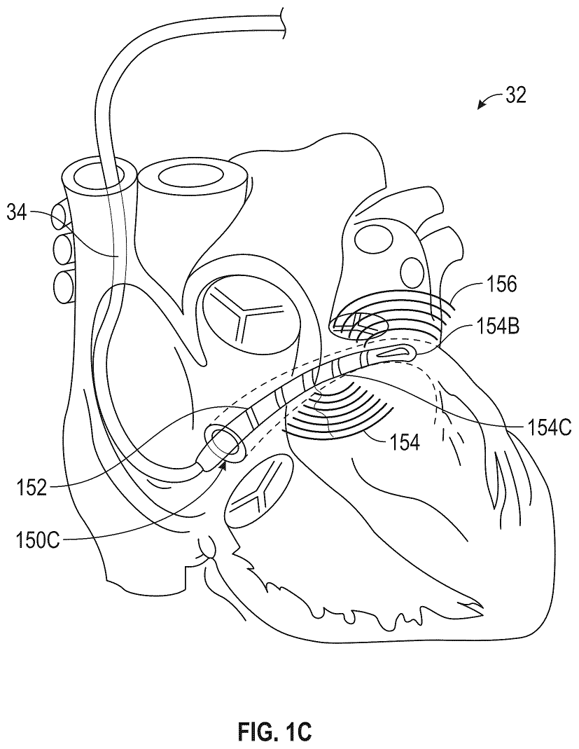

Referring now to FIGS. 1A-1C, FIG. 1A illustrates the practical application in a clinical setting of an exemplary 3D Real-Time Cardiac Mapping System 10 (hereinafter “system 10”). FIGS. 1B-1C depict placement of an exemplary catheter 150B, 150C, respectively, inside the coronary sinus in heart 32 of a patient 12. System 10 is configured for comprehensive cardiac evaluation of patient 12, supporting the use of multiple catheters for enhanced diagnostic versatility, thereby enabling real-time analysis of cardiac wall functions. For example, patient 12 may be undergoing a cardiac treatment (e.g., RFA, TAVR, LAAO, cryoablation, and the like) in a clinical setting with a medical practitioner or user (not shown) operating imagine console 100.

As shown in FIG. 1A, in some embodiments, system 10 may include imaging console 100, system server 102, and external resources 120, in communication with one another via network 106. Imaging console 100 includes display device 101, coupled to catheter 150, and ECG leads 160. As shown in FIG. 1B, catheter 150B may be placed along the endocardial walls of the heart 32 and directed to the coronary sinus 34. Because coronary sinus 34 is anatomically positioned in close proximity to all four heart chambers, clinicians often favor the coronary sinus for placement of diagnostic catheter for stable electrical rhythm monitoring, pacing when required as well as therapeutic purposes. However, placements of catheter 150 outside the coronary sinus and/or outside the heart are possible and have been fully contemplated herein. For example, in some embodiments, catheter 150 may be configured for neural imaging and/or imaging of various internal organs (e.g., intestine, colon, ear, nose, throat, and the like).

In some embodiments, catheter 150B includes electrode pairs 152 and transducer 154. Transducer 154 may sense anatomical structures of the body (e.g., heart 32). Transducer 154 senses by emitting an ultrasound beam having field of view 156. Reflected ultrasound beam energy within field of view 156 reflects off internal cardiac structures. Transducer 154 senses such reflected ultrasound beam signals and transmits the reflected ultrasound signals to console 100 for processing. As shown in FIG. 1C, in some embodiments, catheter 150C may include two transducers 152A, 152B spaced apart from one another. In some embodiments, system 10 utilizes two separate catheters each having a single transducer 154.

In some embodiments, system 10 connects to multiple imaging catheters (e.g., 150B, 150C) and displays both static and live, real-time images of a patient's internal heart structures on display device 101. For example, in some embodiments, console 100 transmits imaging data output by multiple imaging catheters 150 to server 102. Server 102 processes the data and transmits back to console 100 for displaying a unified, real-time image or video stream over a network 106, further enriching the visualization experience.

By collecting imaging data from multiple catheters, or multiple transducers, system 10 may generate detailed renderings of the anatomical structures of the heart in a highly detailed 3D or 4D models of the heart's chambers, enhancing the precision and clarity of these models. Such advanced imaging capability is partly due to System 10's ability to integrate imaging data from various sources and facilitated by localization techniques of the embodiments herein, which are described in detail further below. Such localization techniques refine the accuracy of the 3D/4D renderings in terms of the position relative to other structures and/or devices, and characteristics of the anatomical structures.

In some embodiments, server 102 includes processor 104 in communication with memory 106. Memory 106 may include software code 105. Processor 104 is configured to receive and execute software code 105 for implementing one or more of the embodiments described herein. For example, server 102 may execute code 105 and cause system 10 to connect to ICE ultrasound sources (e.g., catheter(s) 150) and simultaneously present real-time 2D/3D images from one, some, or all sources on a display device (e.g., 101).

In some embodiments, server 102 includes one or more modules for carrying out corresponding functions of the embodiments described herein, which are described in further detail below. For example, in some embodiments, server 102 may include localization module 108, coordinate sync module 110, rendering module 112, object tracking module 114, and predictive module 116. Server 102 may communicate to external resources 120 via network 106.

In some embodiments, network 106 may include, for example, a LAN/WAN connection configured to provide an Internet connection via a hybrid fiber optic (HFC) transmission network, (e.g., Ethernet twisted shielded pair CAT-5, WiFi, premises coaxial cable network, or any other connection capable of establishing an Internet connection). In some embodiments, network 106 may include a wireless network capable of establishing an internet connection (e.g., 5G, LTE, 4G, CDMA, and the like). Network 106 facilitates leveraging external resources 120 for facilitating various functionality, which is described in further detail below.

In some embodiments, external resources 120 may include a remote database and/or access to 3^{rd} party API services that facilitates the integration and interaction between system server 102, and a remote client at imaging console 100, with external systems and resources for enhanced functionality. For example, resources 120 may facilitate connection with a variety of 3^{rd} party API services, enabling the system to leverage external tools and data sources. Such API services could include, but are not limited to, platforms offering advanced AI processing capabilities and predictive analytics tools, which is described in further detail below. In some embodiments, external resources 120 may establish connections with one or more remote databases (not shown), which may be advantageous in augmenting system 10 data handling and processing capabilities. By implementing external resources 120, system 10 may advantageously expand the range of functionalities, such as real-time data analysis, machine learning processes, and sophisticated predictive modeling, which is discussed in further detail below. Such external resources 120 not only enriches the user experience by providing more accurate and efficient outcomes but also enhances the overall system's performance by integrating virtualization techniques for streamlining process efficiency, which is described in detail further below.

One or more components of system 10 (e.g., console 100, processors 104, and/or modules 108, 110, 112, 114, 116) may be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs (e.g. code 105) that are executable and/or interpretable on a programmable system including one or more programmable processor(s) (e.g. 104), which can be special or general purpose, coupled to receive data and instructions from, and to transmit data and instructions to, a storage system (e.g., memory 106), at least one input device, and at least one output device. The programmable system or computing system may include clients (e.g., console 100) and servers (e.g., 102). A client and server are generally remote from each other and typically interact through a communication network (e.g., 106). The relationship of client and server arises by virtue of computer programs running on the respective computers and having a client-server relationship to each other.

Such computer programs, which can also be referred to programs, software, software applications, applications, components, or code, include non-transitory machine readable instructions for a programmable processor, and can be implemented in a high-level procedural language, an object-oriented programming language, a functional programming language, a logical programming language, and/or in assembly/machine language. As used herein, the term “machine-readable medium” (or “computer readable medium”) refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” (or “computer readable signal”) refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

In some embodiments, surgical console 100 may include a plurality of battery packs coupled to charging circuitry and power adapters (not shown) which may be configured to provide electrical power (e.g., AC and/or 55 DC current) to surgical console 100 and catheters 150. In some embodiments, imaging console 100 may include output/input (I/O) ports 103 for data transfer I/O ports 103 may include one or more of: a hardwired telephone jack, USB ports, serial ports, parallel ports, audio ports, video ports, VGA port, a digital video interface (DVI) ports mini-DVI ports, display ports, FireWire ports, Ethernet ports, RJ-11 motor ports and the like. In some embodiments, surgical console 100 may output audio/visual alerts, or audible tones together with visual alerts. For example, audible and visual notifications may be output from built-in speakers, and display 101 may notify users of catheter slips, which is discussed in further detail below

In some embodiments, system 10 via console 100 supports body surface ECG leads data recording, via body surface ECG leads 160. In some embodiments, ECG leads 160 may include 12 body surface ECG sensors. In other embodiments, ECG leads 160 may include less than 12 or more than 12 ECG sensors (e.g., 5, 7, 15, or 20 sensors). In some embodiments, system 10 supports recording of electrode information for any catheter or device with electrodes that may be configured to transmit electrical signals to an EP recording system (e.g., console 100). For example, in some embodiments, when connected to a catheter or device with electrodes, system 10 supports intra-cardiac EGMs measuring voltage of the endo-cardium or epi-cardium. In some embodiments, system 10 records cardiac voltage by connecting electrodes configured for into the intra-cardiac devices and sampling the electrical information. In some embodiments, system 10 may use either WCT or indifferent electrode, positioned in the body at a distance from the heart, as electrical ground to create uni-polar voltage information, which is discussed in detail further below.

In server 102, each module 108, 110, 112, 114, 116 plays a synchronized role in concert with the operation of imaging catheters 150 and console 100. As discussed in detail below, localization module 108 may be tasked with pinpointing the catheter's position, a process that may employ advanced localization techniques like impedance-based localization of the catheter electrodes, electro-magnetic sensor tracking, shape-sensing fiber optics tracking, and/or combination of multiple tracking techniques. Coordinate sync module 110 ensures that the catheters' 150 position aligns with system 10's unified coordinate framework, crucial for accurate anatomical reconstructions. And predictive module 116 applies algorithms to anticipate catheters' 150 optimal positioning, enhancing procedural efficacy and outcome, which is discussed in detail below.

As shown in FIG. 1A, system server 102 includes localization module 108, coordinate synchronization (sync) module 110, rendering module 112, object tracking module 114, predictive module 116, which are configured for implementing the embodiments described herein. As described in further detail below, such modules 108-116 function alone and/or in coordination with one another for implementing the embodiments described herein. In the embodiments described herein, functions described as carried out by one module, may, in other embodiments, be carried out be a different module, or split among various modules. For example, functions described as performed by localization module 108 in one embodiment, may, in another embodiment, be performed by object tracking module 114 and/or coordinate sync module 110, and/or vice versa.

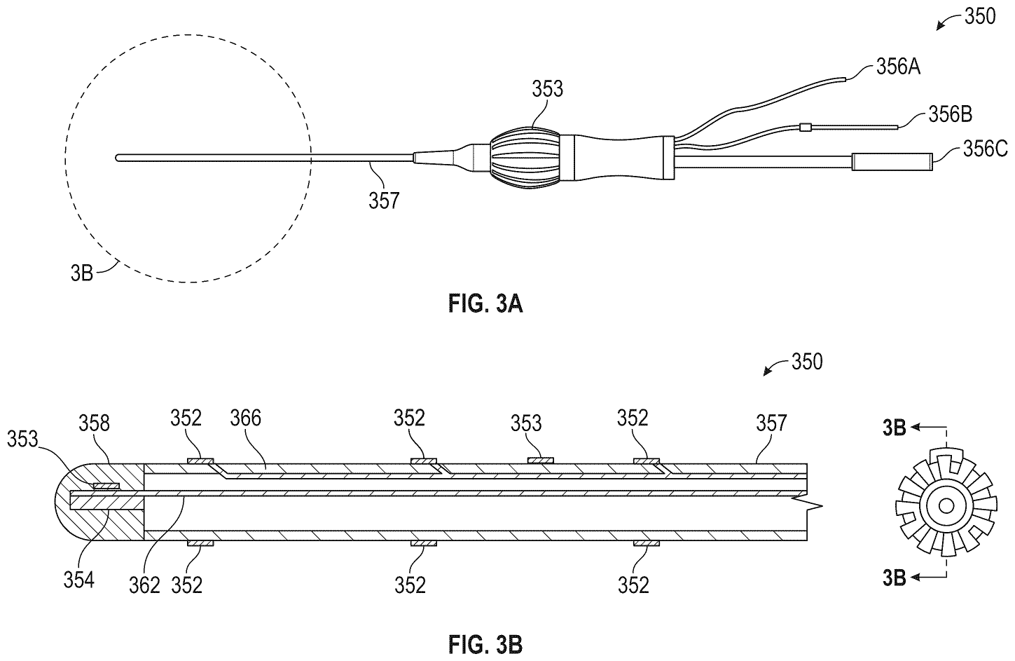

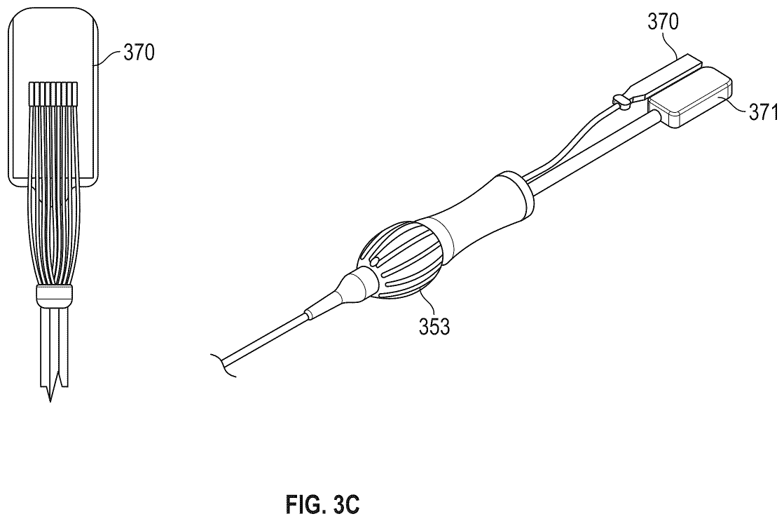

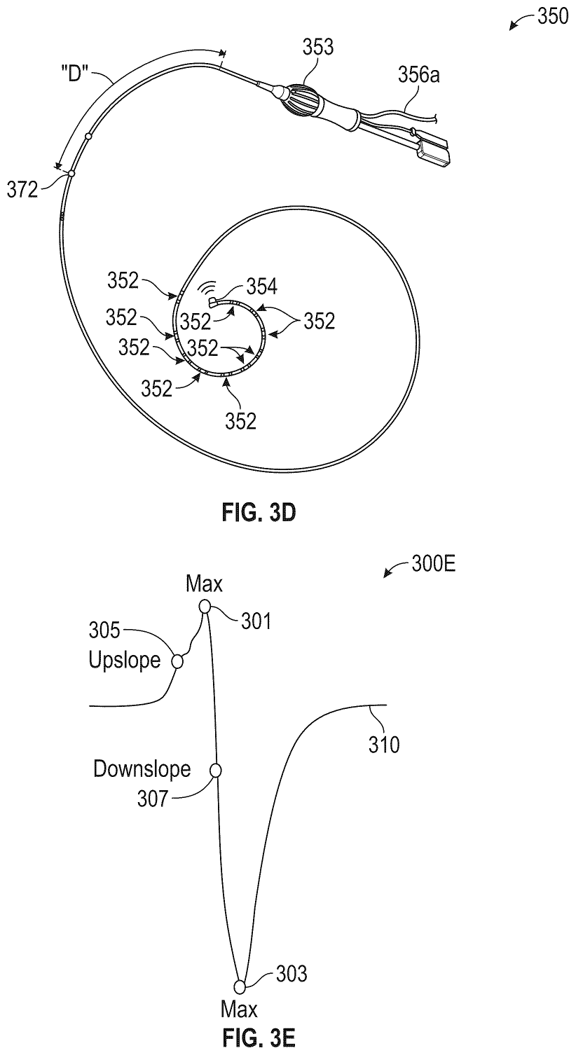

For example, in some embodiments, localization module 108 corresponds to functionality that determines a localization, or localizes, catheter 150 (e.g., by placing magnetic sensors in close proximity to a transducer of ultrasound catheter 150). In some embodiments, such localization may be implemented by using shape sensing fiber optic wire embedded within catheter 150 shaft and/or splines, which is discussed in further detail below in FIG. 3B. In other embodiments other localization techniques for localizing the transducer position and orientation may be implemented, which is described in further detail below.

In some embodiments, system 10 may identify anatomical structures inside heart 32 by analyzing imaging data from catheter(s) 150. In some embodiments, such cardiac anatomical structures may include one or more of lower chambers (i.e., left and right ventricles), upper chambers (i.e., left and right atria), cardiac walls (i.e., the muscular walls that separate the heart's chambers, including the interventricular septum between the ventricles), heart valves (e.g., mitral, tricuspid, aortic, and pulmonary valves), coronary arteries and veins, pulmonary veins and arteries, septa (e.g., atrial and ventricular septa), aorta, ostia, superior and inferior vena cava, as well as other clinically relevant anatomical cardiovascular structures, which are known to a person having ordinary skill in the art.

In some embodiments, identifying anatomical structures may include indicating, with a graphic user interface overlay, the contours on the ultrasound images displayed on display device 101. Such indications may be selected by the user and/or predicted by system 10, via prediction module 118. In some embodiments, system 10 may reconstruct the cardiac anatomy by creating 3D images on the system's monitor (e.g., display device 101). In some embodiments, system 10 may support static anatomical reconstruction, (i.e., anatomical reconstruction corresponding to specific time along the cardiac cycle). In some embodiments, system 10 may support dynamic anatomical reconstruction, where the chamber reconstruction corresponds to the cardiac cycle, capable of representing cardiac wall movements.

In some embodiments, system 10 significantly enhances the visualization and manipulation of medical devices like catheters and sheaths within the heart via localization module 108. In some embodiments, localization module 108 may, for example, implement electromagnetic-based localization via trackingDegrees-of-Freedom (DOF), and/or impedance-based localization by analyzing transmissions from catheter electrodes, body surface patches and/or shape sensing fiber optics, which are described in detail further below. Such localization facilitates precise rendering of catheters, sheaths, or devices embedded inside heart 32, thereby enabling accurate positioning of such images relative to a reconstructed cardiac anatomy. System 10 may display such images in real time 2D and/or 3D views, offering comprehensive visual insights for medical professionals.

Described in further detail below, in some embodiments localization module 108 and coordinate synch module 110 operate to synchronize objects and devices rendered on the anatomical reconstruction within a unified 3D coordinate system. Such synchronization ensures that the positions of catheter(s) 150 or other devices are accurately represented in relation to the cardiac anatomy, facilitating precise and informed medical interventions. In some embodiments, such renderings may be displayed by rendering module 112, which transforms data output by catheter 150 into visual representations. Rendering module 112 operates as the integrative visual output center of system 10. Rendering module 112 collaborates closely with other system 10 modules (e.g., 108, 110, 114, 116) to generate real-time and static 2D/3D visualizations of the heart's internal features.

For example, in some embodiments, rendering module 112 receives anatomical and positional data coordinate sync module 110, object tracking module 114 and localization module 108, which provides precise spatial information. Rendering module 112 continuously communicates with the coordinate sync module 110 to ensure that all images are accurately aligned within a unified spatial framework. For example, object tracking module 114 contributes of surgical instruments within the heart, while the predictive module 116 offers foresight into potential movements of cardiac structures based on past and present data trends. Working in unison, such modules (e.g., 108, 110, 114, and/or 116) enable rendering module 112 to output detailed, real-time 3D cardiac renderings advantageous for diagnostic and therapeutic procedures.

In some embodiments object tracking module 114 provides dynamic tracking and surgical device identification. For example, object tracking module 114 identifies devices placed within the heart, particularly when the position of such device intersects with transducer 154 field of view 156. Such identification may be performed by utilizing advanced image processing techniques (e.g., a convolutional neural network predictive model), which may be further enhanced by the localization data when an embedded device is being localized by localization module 108. Such identification is advantageous for ensuring accurate placement and movement of embedded devices within the heart.

In some embodiments, coordinate sync module 110 continuously, substantially continuously, or repetitively (i.e., digital sampling) associates positions representing embedded devices' localization within a 3D coordinate system. Such positions are utilized to contribute to the reconstruction of cardiac anatomy. Since all trackable objects are localized within the same coordinate system, the sampled positions may identify areas categorized, for example, as either being in a blood pool or in contact with anatomical structures, such as cardiac wall tissue. Such identification of blood pool or anatomical structures may be advantageous in contributing to the cardiac anatomy reconstruction, which may originate from various sources, including ultrasound or catheter positions, and is discussed in further detail below.

In some embodiments, predictive model 116, may advantageously facilitate the integrative core of the system 10, collaborating with each, some, or all modules (e.g., 108, 110, 112, 114,) to enhance system 10 capabilities. For example, in some embodiments, predictive model 116 operates with localization module 108 to anticipate and refine the positioning of imaging catheter(s) 150, adapting to the patient's unique cardiac anatomy. In some embodiments, via connection with coordinate sync module 110, predictive model 116 aligns such predictions within the established 3D framework, ensuring that the rendered images by rendering module 112 accurately reflect the heart's structure and dynamics, which is described in detail further below.

In some embodiments, predictive module 116 leverages the data processed by the object tracking module 114 to forecast the movement of the catheter within the cardiac chambers, aiding in maintaining continuous and precise imaging. The external resources 120, which include access to expansive databases and cutting-edge AI processing tools, may be utilized by predictive module 116 to enhance its analytical models, providing a deep learning aspect to system 10 operations, which is discussed further below. Such comprehensive interaction allows predictive module 116 not only to react to the current state but also to effectively predict future states, thus optimizing the entire imaging and treatment process and advantageously harnessing the combined power of internal modules and external AI enhancements.

Advantageously, system 10 may simultaneously operate multiple surgical tools and/or catheters of different types, or the same type. Such catheters are integral to structural heart procedures and electrophysiology and serve both diagnostic and therapeutic functions. For example, in structural heart procedures, the emphasis is on anatomical diagnosis, where mechanical motions of cardiac tissues are analyzed. In electrophysiology, catheters go further, evaluating cardiac functions by measuring tissue voltage and activation to guide treatment decisions. Clinicians frequently adjust catheters to capture the necessary imaging angles, vital for guiding trans septal puncture and catheter position within the heart.

The coronary sinus, often hosting linear catheters equipped with electrodes, is advantageous for diagnosing arrhythmias and assessing atrial and ventricular electrical patterns. For example, FIG. 1B depicts catheter 150B as a 10-pole (pole number means number of EGM electrodes) catheter with transducer 154B positioned at, or substantially at, the distal end of catheter 150B, or close to the distal end of catheter 150B. In FIG. 1B, imaging catheter 150B sensing component (e.g., transducer 154B) includes field of view 156 pointing in the general direction of the left atrium. In some embodiments, other placements are suitable. For example, the aortic valve may be suitable for placement based on the procedural requirements of a particular cardiac treatment (e.g., RFA, TAVR, LAAC, cryoablation, and the like).

In some embodiments, as shown in FIG. 1C, catheter 150C may be configured with transducer 154B oriented in the general direction of the left atrium, and transducer 154C may be oriented in an opposite direction respective to the orientation of transducer 154B, for example, towards the right ventricle. Such placement may be advantageous for reconstruction and renderings of 3D/4D mapping of the cardiac structures in heart 32, which is discussed in detail further below.

Placing catheter 150B, 150C (collectively, “catheter 150”) in the coronary sinus (CS) vein through the CS ostium, in accordance with some embodiments herein, may be achieved without using a sheath due to self-deflection mechanism, which, in some embodiments, is part of catheter 150's design (e.g., torque-ability and adequate diameter), which are discussed in detail further below. When placing the imaging source in the CS vein, transducer 154 may be positioned and torqued to provide images of the right atrium, atrial septum, left atrium, left ventricle and right ventricle. With catheter 150 inserted into the CS vein, a greater stability is achieved compared with the alternative of positioning a catheter in the cardiac chamber where any slight movement changes the transducer position and angle, forcing the physician to manually adjust or hold the catheter throughout the procedure.

In some embodiments, catheter 150 may have more than two transducer 154 transducers (e.g., three, four, five, or more) allowing multiple 2D and 3D images captured simultaneously. Such transducers may be positioned along the same plane across catheter 150 surface or facing in opposite direction when embedded in the catheter at either parallel or staggered positions relative a preceding or subsequent transducer. Embedding more than one transducer allows for different perspective on the same invasive tool positioned in one of the cardiac chambers or anatomical structure or visually monitoring in real-time two separate anatomical structures, cardiac chambers, other catheters or tools positioned in the cardiac chambers or any combination of the abovementioned, which is discussed in further detail below.

In some embodiments, electrodes sensing the electrical conductivity of heart 32 advantageously serve multiple purposes, including sensing electrical activity within the heart and measuring tissue impedance. The importance of measuring the voltage from multiple electrodes from within the heart is advantageous in procedures where electrical conductivity of the cardiac chambers is relevant for diagnosis and treatment. Due to the CS vein unique anatomy, a properly placed catheter (e.g., 150) may sense and track signal sequence and morphology of the right atrium, left atrium and/or right ventricle, which is advantageous for electrophysiology monitoring.

Impedance in biological tissues is a measure of resistance to electrical current flow. By applying a small, known electrical current through one electrode and measuring the resulting voltage at another electrode, system 10 may determine the tissue's impedance. Tissue characterization considers different types of cardiac tissues (e.g., healthy tissue, scarred areas, and/or blood pools) have distinct impedance characteristics. By measuring impedance, system 10 may differentiate between such cardiac issue types, which is advantageous for procedures like cardiac ablation, where identifying the target area accurately is advantageous.

Impedance/voltage measurements may also be advantageous in determining the contact quality between catheter 150 and the cardiac tissue of heart 32. For example, a high impedance may indicate poor contact or that electrode 152 is in blood, while lower impedance suggests good tissue contact. During procedures, real-time impedance monitoring allows for continuous assessment of catheter-tissue contact, ensuring effective and safe energy delivery during ablation or accurate signal recording during diagnostics.

Apart from impedance, electrodes 152 also record the heart's electrical activity. Such electrical activity data is advantageous in electrophysiology studies to understand the origins of arrhythmias and to guide therapeutic interventions. Some embodiments include electrical activity integration with imaging. For example system 10 may analyze impedance data from electrodes 252 and enhance GUI renderings of catheter 250 interactions with cardiac structures, which is discussed in detail further below.

Accordingly, in some embodiments, catheter 150, 250 may be configured for unipolar voltage measurements, wherein one electrode on the catheter measures the voltage relative to a reference electrode or the Wilson-Central-Terminal (WCT) that is derived from the body surface electrode readings output by ECG leads 160. In other embodiments, catheter 250 may be configured for bipolar voltage measurements, wherein two electrodes on catheter 250 are configured for measuring the voltage difference between such two electrodes. Bipolar voltage measurements may be advantageous for detailed analysis of electrical activity within specific heart regions.

For clarity, the difference between unipolar and bipolar voltage information lies in how the electrical signals are measured by electrodes. Uni-polar voltage recording provides a broader view of the electrical activity but may be more susceptible to interference, noise and electrical activity at a distance from the electrode, i.e. far-field voltage. Bipolar voltage recordings involve measuring the voltage difference between two closely spaced electrodes (e.g., electrodes 256). Bi-polar voltage recordings localized information about the electrical activity and may be less affected by noise or far-field activation voltage.

Thus, in some embodiments, catheter 150, 250 has four or more ring electrodes either uniformly spread, or in a bipolar measurement configuration where every two electrodes are close to each other with larger distance between each pair. In some embodiments, transducer 154s 254 may be located close to the catheter tip or positioned between the electrodes. For example, transducer 254 may be placed at the tip of an eight-pole catheter or at the center between the four most distal electrodes and the four most proximal electrodes. For example, catheter 250D includes transducer 154 positioned at, or near, the tip of the catheter and another transducer 154 positioned at or near the center of the catheter body between the ten most distal electrodes and ten most proximal electrodes, facing in opposite direction in a duo-deca diagnostic CS catheter. In some embodiments, catheters 150 may include four or more voltage ring electrodes with either uniform distance between electrodes or, in some embodiments, bi-polar configuration. In some embodiments having bipolar configurations, at least one or more pairs of adjacent electrodes are placed closer together relative to other pairs, with a greater distance maintained between such pairs for enhanced specificity in electrical activity detection within the heart's targeted regions, which is described in detail below.

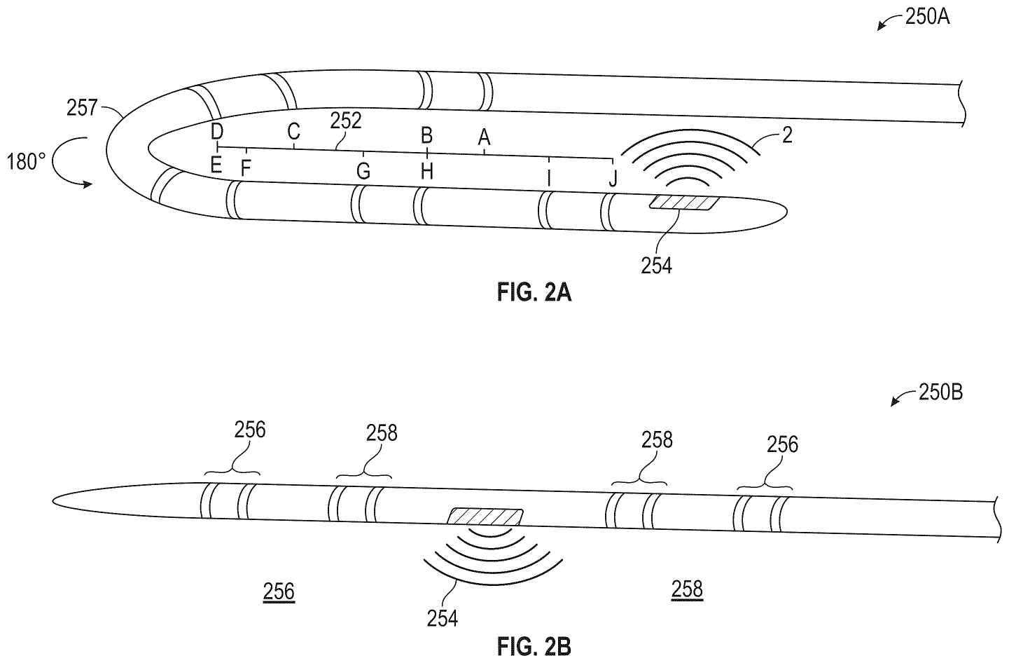

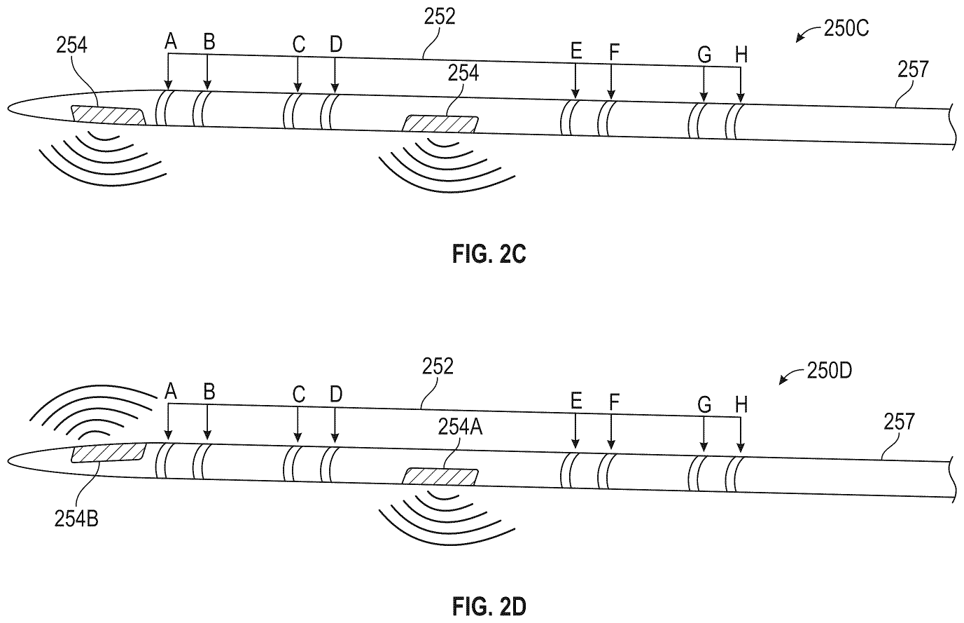

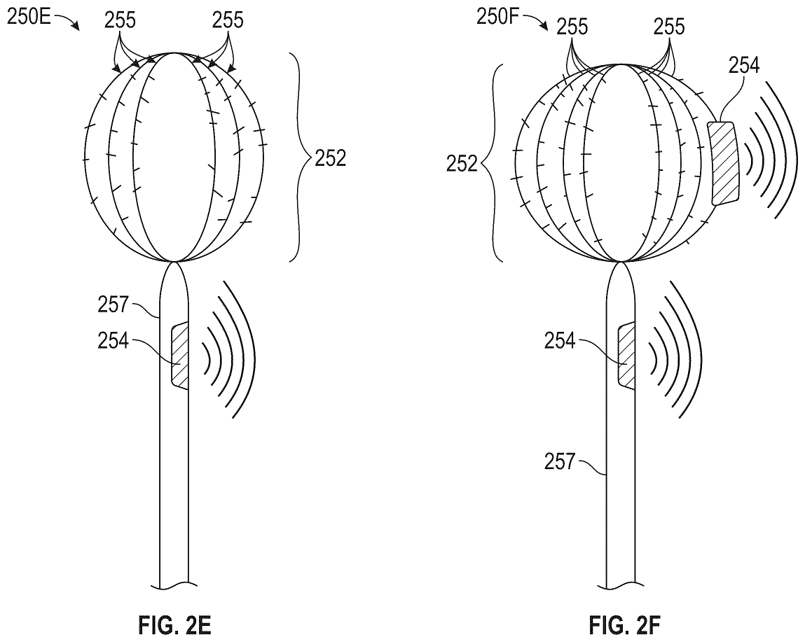

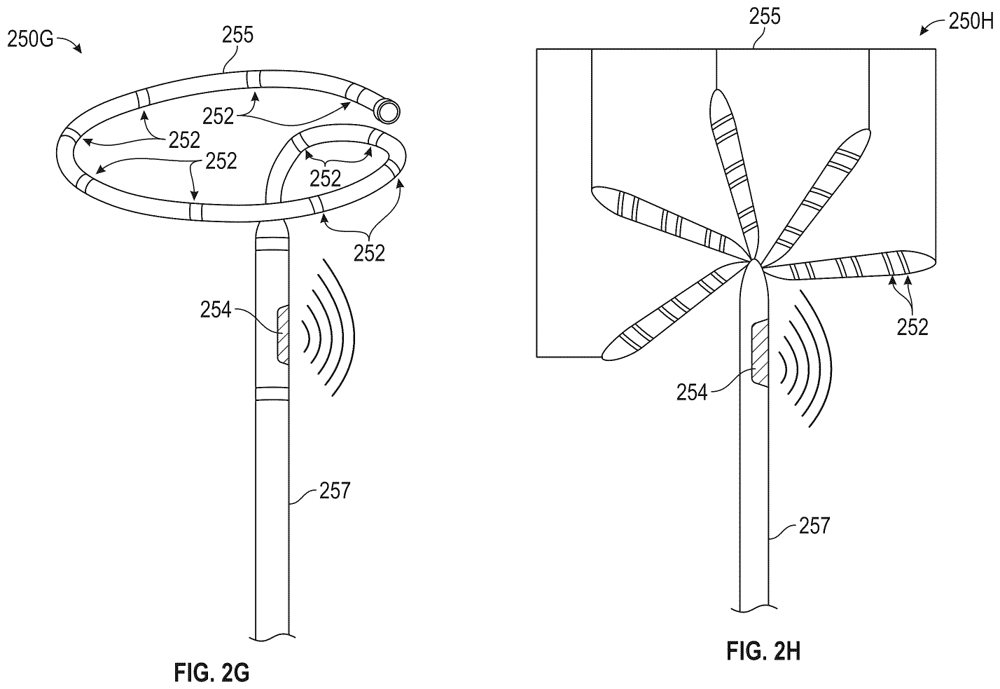

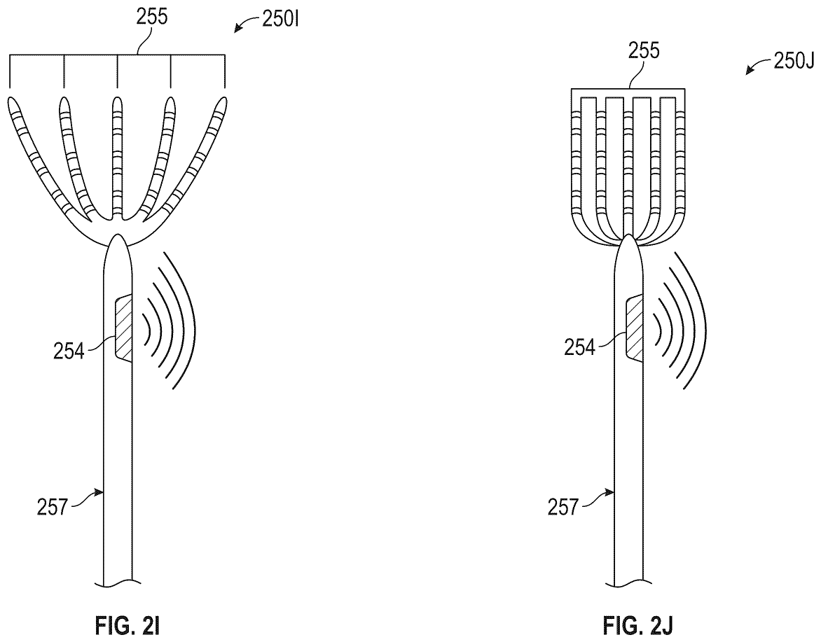

Referring now to FIGS. 2A-2F, in some embodiments, system 10 via console 100 may simultaneously analyze data from one, or more than one, of the same, or different, catheter(s) 250A-250F. For example, FIG. 2A depicts a linear catheter 250A in a deflected position. In some embodiments, catheter 250A is capable of up to 180-degree deflection. For example, catheter 250A may include a deflection mechanism, which may be actuated by a deflection handle (e.g., shown in FIG. 3A). Such deflection may be uni-directional, or in some embodiments, bi-directional. In some embodiments, such deflection may be greater than 180 degrees of deflection, and less than, or equal to, 360º of deflection.

In some embodiments, catheter 250A includes 10 electrodes 252A-252J. Electrodes 252 may be paired (e.g., 252A, 252B) with a shorter distance between electrodes in each pair compared to longer distance between pairs of electrodes (e.g., electrode pair 252A, 252B, cf. electrode pair 252D, 242C), and transducer 254. In some embodiments, 5DOF/6DOF magnetic sensors (not shown) are added along catheter shaft 257 and configured for spatial tracking, which is discussed further below. In some embodiments, transducer 254 may be positioned substantially at the distal end of catheter 250A, or close to the catheter distal end (i.e., the catheter tip).

In some embodiments, electrodes 252, 258 may include one or more of surface electrodes, tip electrodes, and/or ring electrodes. Such surface electrodes may include conductive pads or rings placed along the catheter's surface. Surface electrodes come into direct contact with the heart tissue and are used to record electrical signals from the myocardium. Tip Electrodes are located at the distal end of the catheter, or substantially at the distal end. Tip electrodes may aid in diagnostic and therapeutic procedures, such as ablation, to deliver electrical energy or record signals from specific cardiac regions. Ring electrodes correspond to circular electrodes spaced along the catheter body. Ring electrodes are advantageous for mapping electrical pathways across a larger area of tissue.

As shown in FIG. 2B, in another embodiment, catheter 250B may include a linear deflectable catheter with 8 electrodes, where every 2 electrodes are paired with a shorter distance between each pair and longer distance between pairs of electrodes similar to, or the same as catheter 250A, supra. In some embodiments, electrodes 252 may be spaced apart non-uniformly, and may vary a distance between coupled electrode pairs. For example, such variance may include between 1 mm to 4 mm and the distance between pairs of electrodes ranges between 4 mm to 10 mm or more. In some embodiments, the distance between pairs of electrodes may range between 3 mm to 11 mm, 2 mm to 12 mm or 1 mm to 13 mm, for example. In some embodiments, distal 4 electrode pairs 256 and proximal 4 electrodes pairs 258 may be interspaced and have sufficient spacing to accommodate one or more transducers. For example, as shown in FIG. 2B, ultrasound transducer 254 may be positioned at a relative center between distal 4 electrodes 256 and proximal 4 electrodes 258.

FIG. 2C depicts catheter 250C, which is an embodiment of catheter 250B, wherein similarly labeled parts and numbers correspond to similar features having similar functionality. Catheter 250C, in some embodiments, includes 8 electrodes where every 2 electrodes are paired with a shorter distance between each pair and longer distance between pairs of electrodes, similar to, or the same as described above. As shown, both transducers 254 are oriented pointing to the same direction.

FIG. 2D depicts an embodiment wherein linear catheter 250D includes 8 electrodes where every 2 electrodes are paired with a shorter distance between each pair and longer distance between pairs of electrodes. The distal 4 electrodes and the proximal 4 electrodes have larger distance between one another to accommodate transducers. Transducer 254A is placed at the center between the 4 distal electrodes and the 4 proximal electrodes. Transducer 254A, 254B ore oriented pointing to different directions, for example, in opposite directions. In some embodiments, such orientation may be adjusted via console 100.

Referring now to FIG. 2E-2J, FIG. 2E depicts catheter 250E, which is a deflectable basket-shaped multi-electrode mapping catheter with transducer 254 embedded at shaft 257, after the proximal end of the basket structure. Catheter 250E includes 6 splines 255 with 6 electrodes 252 on each spline 255. Catheter 250E includes transducer 254 on shaft 257 positioned on or near the proximal end of shaft 254.

In some embodiments, catheter 250E may include more or less electrodes 252 on spline(s) 255, more or less number of splines 255, and uniform or non-uniform spacing between electrodes 252. For example, one embodiment may include catheter 250E having 4 splines 255 with 8 electrodes 252 on each spline 255, resulting in a 32-pole catheter. In some embodiments, the spacing may be substantially uniformly 6 mm between electrodes while an 8 spline basket with 12 electrodes each and uniform 2 mm spacing results in 96 electrodes in total.

As shown in FIG. 2F, in one embodiment, catheter 250F may be a deflectable basket-shaped multi-electrode mapping catheter with transducer 254 embedded in one of the catheter splines 255. For example, an individual spline 255 may have no electrodes and instead embed transducer 254, as shown. Catheter 250F may, when collapsed, utilize transducer 254 located on one of the splines as standalone ICE ultrasound imaging catheter. When fully deployed, to obtain a basket shape, catheter 250F may collect impedance/voltage data using electrodes 252.

In some embodiments catheter 250F may include a bi-directional deflection mechanism (not shown) and transducer 254 located on shaft 257 following the proximal end of the basket (as shown). Catheter 25OF electrodes 252 may include an electrode spacing ratio of 2-4-2 relative to catheter pairs. In some embodiments, catheter 250F may include 8 splines with 8 electrodes on each spline and no transducer on the splines.

FIG. 2G depicts a circular or loop shaped mapping catheter 250G, in accordance with some embodiments. Catheter 250G includes 10 electrodes 252 with 2-6-2 spacing on spline 255, uni-directional deflection mechanism and transducer 254 located on shaft 257 following the loop star. Another embodiment uses a deflectable circular-shaped catheter with 10-20 electrodes on the catheter loop with uniform or non-uniform spacing and transducer 254 positioned in shaft 257 where the loop starts.

In some embodiments, catheter 250G may include a loop mechanism that may maintain a fixed size or be adjusted for flexibility. Such variable loop functionality of catheter 250G allows the diameter of the loop to be “shrunk,” enabling precise manipulation and navigation within the cardiac anatomy. In some embodiments, catheter 250G may be configured for a dual loop configuration with a reduced loop diameter, enhancing the versatility and effectiveness in accessing and treating intricate areas of the heart. Such adjustable loop control is advantageous for cardiac procedures requiring nuanced control and spatial adaptability.

FIG. 2H depicts a flower shaped mapping catheter 250H, in accordance with some embodiments. Catheter 250H includes 5 splines 255 with 6 electrode pairs 252 with uniform 2 mm spacing on each spline 255. Catheter 250H may include a uni-directional deflection mechanism (not shown) and ultrasound transducer 254 located on shaft 252 following the flower splines origin. 250H tip is flower-shaped with splines originating from the same center and pointing perpendicular to the catheter shaft in evenly spaced angle. i.e., 5 splines will have 72 degrees between each spline and 8 splines will have 45 degrees between splines. In some embodiments, various number of electrodes may be placed on each spline with uniform or non-uniform spacing. Transducer 254 may be positioned on shaft 257 connecting all the splines, as shown in FIG. 2H.

FIGS. 2I-2J depict spline shaped mapping catheter 250I, 250J, respectively, in accordance with some embodiments. Catheter 250I, 250J is a flat spline configuration where the splines are leveled on the same plane and pointing to the same direction. As shown in FIG. 2I, in catheter 250I all, some, or at least one spline 255 is/are separated from one another and taper to be positioned in parallel, or substantially parallel, to all, some, or at least one of other splines 255. Such splines may include a close-ended configuration, as shown in FIG. 2I, where splines 255 are substantially parallel and connected at the distal end of each spline 255. Or, in some embodiments, splines 255 may be open-ended, as shown in FIG. 2J, having no connecting portion on the distal ends of splines 255. In some embodiments, transducer 254 may be placed on shaft 257 connecting splines 255. In some embodiments, the splines plane is 20 degrees to the shaft plane. In some embodiments, the splines plane and shaft planes intersect at more than 20° degrees or less than 20° degrees (e.g., 10°, 15°, or 30°) In some embodiments, catheters 250I, 250J include 5 splines with 6 electrodes with uniform 2 mm spacing on each spline.

In some embodiments, catheters 150, 250 are configured for noise reduction and may include a signal reference electrode (not shown in FIG. 2) positioned at a distance of 20-40 cm from the catheter handle (not shown in FIG. 2). Discussed in further detail below, such signal reference electrode may be used as a ground, differentially processing the electrical signals collected by catheter 150, 250. Such signal reference electrode filters out ambient electrical noise, which can stem from various sources, such electromagnetic interference from lab equipment and/or ambient radiation. By referencing signals against such ground electrode, system 10 may effectively isolate and subtract noise from the cardiac signals, resulting in a clearer, more accurate representation of the heart's electrical activity. Such noise cancellation is advantageous for the precision required in cardiac diagnostics and interventions, ensuring that the data collected is reflective of the patient's physiological state rather than the surrounding electronic environment. While linear CS catheters 250A, 250B may advantageously implement such the reference electrode, in some embodiments, catheter 250I, 250J may advantageously omit such reference catheters, which is discussed in further detail below. Other noise attenuation protocol may be implemented and have been fully contemplated herein.

For example, some embodiments implement adaptive filters that adjust parameters in real-time based on the noise characteristics, which may change dynamically during a cardiac treatment or imaging procedure. Signal averaging techniques to reduce noise that is random and not correlated with the cardiac signal may be implemented by averaging multiple signal cycles. Noise may be minimized relative to the true signal. Some embodiments may utilize digital signal processing (DSP) applied to raw signals to detect and remove frequencies that are known to be noise. Other noise reduction techniques may include shielding and rounding by enhancing the catheter's shielding and providing additional grounding to the system to prevent the introduction of electrical noise. Wavelet transformation may distinguish between noise and the cardiac signal in the frequency domain. Predictive modeling using machine learning trained on large datasets to recognize and filter out noise. One or more of the foregoing protocols may be integrated into the system 10 modules (108-116), software (105) or hardware (102) to support the signal reference electrode's function and ensure the integrity of the cardiac signals obtained during diagnostic and therapeutic procedures.

Referring now to FIGS. 3A-3D in conjunction with FIG. 1-2, FIG. 3A depicts a cut-out view showing detailed structure of catheter 350 for use in system 10. Catheter 350 is an embodiment of catheters 150, 250, wherein similarly labeled parts and numbers correspond to similar features having similar functionality. As shown in FIGS. 3A-3B, in some embodiments, catheter 350 may be configured as a linear catheter and includes shaft 357, deflection handle 353, and reference line 356A, power line 356B, and data line 356C.

In some embodiments, shaft 357 may be deflectable, allowing for maneuverability within the cardiac anatomy. In some embodiments, such rotatable actuation may cause up to 180 degrees of deflection in shaft 357. In some embodiments, such deflection may include more than 180 degrees, for example up to 360 degrees. For example, deflection handle 353 may rotatably actuate deflection of shaft 357, thereby controlling a direction of catheter tip 358.

For example, in some embodiments, handle 353 allows for precise directional control of catheter tip 358, enhancing navigability within complex cardiac structures. As mentioned above, such deflection maybe unidirectional, allowing movement in a single plane, or, in some embodiments, bidirectional, providing greater flexibility with the capability of at least 180° to 360° of deflection, thus offering a full circumferential maneuverability. Such deflection mechanism is incorporated within catheter's handle 353. In some embodiments, handle 353 may include a rotating knob, or a plunger system in other embodiments. Such configurations offer different tactile experiences and levels of control, tailored to the procedural needs and the user's preference.

In some embodiments, shaft 357 may include tip 358, ultrasound transducer 354, flex circuit 362, EGM electrodes 352, and EGM line 366. Ultrasound transducer 354 may be positioned towards tip 358 and configured for capturing real-time images from within heart 32. As shown in FIG. 3B, in some embodiments, tip 358 may be positioned at the distal end of shaft 357. Tip 358 may include an atraumatic soft tip that advantageously minimizes tissue damage upon contact with cardiac tissue.

In some embodiments, transducer 354 is communicatively coupled to system 10 via flex circuit 362 advantageously implementing the integration of imaging and electrophysiological functionalities in system 10. In some embodiments, flex circuit 362, may include a fiber optic cable configured for flexibility and durability, facilitating navigation of the intricate cardiovascular pathways. The fiber optic cable of flex circuit 362 may be coupled to shaft 357 and configured to carry optical signals (i.e., light), which are altered by the bending and twisting as the fiber optic cable moves, thus enabling shape sensing capabilities within the heart's chambers.

Such changes in the light signal provides real-time data on the catheters' 350 position and shape, which is advantageous for accurate placement and navigation during cardiac procedures. For example, when fiber optic shape sensing cable is bent or twisted as shaft 357 is bent or twisted by actuating handle 353, fiber optic shape sensing cable of flex circuit 362 alters the path and characteristics of the light traveling through the fiber optic conduit. By measuring changes in optical parameters of the signal output of fiber optic shape sensing cable of flex circuit 362 (e.g., light intensity, phase, or polarization at various points along the cable), system 10 may determine fiber optic shape sensing cable (and thus the catheter's) shape and position.

Advantageously, the fiber optic cable of flex circuit 362 is highly sensitive and may detect difficult to observe changes in shape or position. Such sensitivity makes shape sensing fiber optic cables advantageous for cardiac imaging where high precision is advantageous when navigating catheters in complex anatomical pathways. For example, in some embodiments, as the catheter navigates through heart 32, fiber optic cable of flex circuit 362 continuously relays information about its shape and position. Such shape and position data may be processed by system 10 to reconstruct the catheter's 3D configuration in real time.

Advantageously, fiber optic shape sensing of flex circuit 362 provides direct physical data about the catheter's shape and position. Such direct physical data may result in more accurate and immediate feedback when compared to impedance measurements alone. Fiber optic shape sensing is further advantageous in environments where electrical properties are variable or hard to interpret. Some embodiments implement, fiber optic shape sensing in addition to impedance-based sensing and/or 5DOF/6DOF magnetic sensing. Such multi-modal approach may be advantageous for enhancing the accuracy and reliability of catheter navigation and object detection in real time.

In some embodiments, catheter 350 includes EGM wire 366, which communicates a signal that contains data utilized to record electrical activity of heart 32. As shown in FIG. 3C, EGM wire 366 runs from an EGM electrodes 352 in shaft 357 to pin-box connector 370. Pin-box connecter 370 may be configured for electrical signal transmission.

In some embodiments, catheter 350 may include voltage reference electrode 372, which is shown in FIG. 3D. In some embodiments, voltage reference electrode 372 may be advantageously positioned at spacing, D, or spaced apart from strain relief valve 355, which is part of catheter 350's entry point into the body of patient 12. In some embodiments, such advantageous spacing (D) may be in a range between 20 cm to 40 cm.

Spacing (D) facilities voltage reference electrode 372 being positioned in a vasculature at an advantageous distance below the diaphragm, which ensures voltage reference electrode 372 is sufficiently distant from the heart. Such positioning provides a robust unipolar reference for body surface ECG and intra-cardiac EGM unipolar voltage signals, thereby enhancing the accuracy and reliability of body surface ECG and intra-cardiac EGM recordings. Because minimizing electrical interference from catheter 150, 250 and other nearby medical devices provides a robust reference point for electrical signals such positioning of voltage reference electrode 372 is especially well suited for system 10 operations. However, other embodiments may include a spacing of less than 20 cm or more than 40 cm. For example, in some embodiments, 10 cm or in other embodiments, 55 cm.

In some embodiments, because voltage reference electrode 372 provides a grounding point against which the electrical potentials on the body's surface are measured, such body surface ECG data may be leveraged for advantageously capturing the heart's electrical activity from different angles and positions on the body. In the case of intra-cardiac EGM, voltage reference electrode 372 may advantageously aid in the measurement of electrical signals from within the heart. As catheter electrodes (e.g., 352) move through different cardiac chambers, voltage reference electrode 372 ensures a consistent baseline for all, substantially all, or some measurements, enhancing the clarity and reliability of intra-cardiac electrical recordings. And the advantageous placement (D) of voltage reference electrode 372 aids in reducing electrical noise and artifacts caused by close, local body activities or other medical equipment.

In some embodiments, voltage reference line 356a may be coupled to voltage reference electrode 372 and communicate voltage reference signals to system 10. Voltage reference electrode 372 may be coupled to system 10 via line 356a, which may reside on a dedicated circuit to system 10. Doing so may provide several advantages over integrating with all electrodes on the same circuit. A separated reference line 356a may reduce electrical noise interference from electrodes' 352 signals, leading to cleaner and more accurate data transmission. Because line 356a is well suited for maintaining the integrity of the reference signal output by voltage reference electrode 372, reference voltage data ensuring it is not affected by the cumulative impedance or signal alterations that could occur in a shared circuit. Line 356a offers a stable baseline for comparative measurements, crucial for accurate unipolar recordings. If there is a malfunction or degradation in the catheter 250 circuitry, the separate line for voltage reference electrode 372 remains unaffected, preserving its functionality for consistent measurements. Moreover, having a separate circuit via line 356a allows for independent control and adjustment of the voltage reference electrode's 372 signal, which can be tailored for specific diagnostic or therapeutic needs.

Thus, implementing voltage electrode 372 may advantageously provide cleaner and more interpretable ECG and EGM signals. Such setup is particularly beneficial for unipolar voltage measurements, providing a stable and distant reference point that ensures the reliability and clarity of cardiac electrical signal recordings. For unipolar signal recording, the voltage at a specific point (such as an electrode on a catheter in the heart) is measured relative to a reference point. The reference electrode serves as the reference point and stable baseline and may also be useful for QRS complex analysis, described below.

Referring now to FIG. 3E, FIG. 3E depicts a diagram 300E of an exemplary Intracardiac ECG signal 310. ECG signal 310 corresponds to ECG data output via voltage reference electrode 372. For clarity, ECG signal 310 is a representative example of an intracardiac signal derived from electrode 372, which represents the electrical depolarization that leads to the contraction of the heart. The representative signal consists of three main deflections: the initial upstroke, similar to an R wave on a body surface ECG, the downstroke, similar to a Q wave, and the recovery back to baseline, similar to a S. The duration, amplitude, and morphology of the waveform of ECG signal 310 provides diagnostic information about cardiac health and are used to identify various cardiac conditions.

Accordingly, system 10 may be configured to analyze ECG signal 310, which may be isolated from a single or multiple intra cardiac signals from catheter(s) 350 that is referenced to a stationary fiducial point, preferably a catheter placed in the CS. ECG signals 310 provides metrics that provide a consistent, reliable temporal reference that does not change between heartbeats throughout a cardiac treatment. ECG Signal 310 may be associated with one or more metrics associated with the structure of the intracardiac signal on electrode activation. As shown in FIG. 3E, such metrics may include one or more of: max peak 301, minimum peak 303, upslope 305 (max dV/dT), and/or downslope 307 (i.e., max negative dV/dT). In some embodiments, such metrics may be displayed in real time via one or more overlays presented by GUIs of system 10, which is discussed in further detail below. In some embodiments, ECG signal 310 and corresponding metric data may be integrated with spatial and temporal mapping to provide a comprehensive view of the heart's electrical activity, aiding in the diagnosis and treatment of arrhythmias, which is discussed in detail further below.

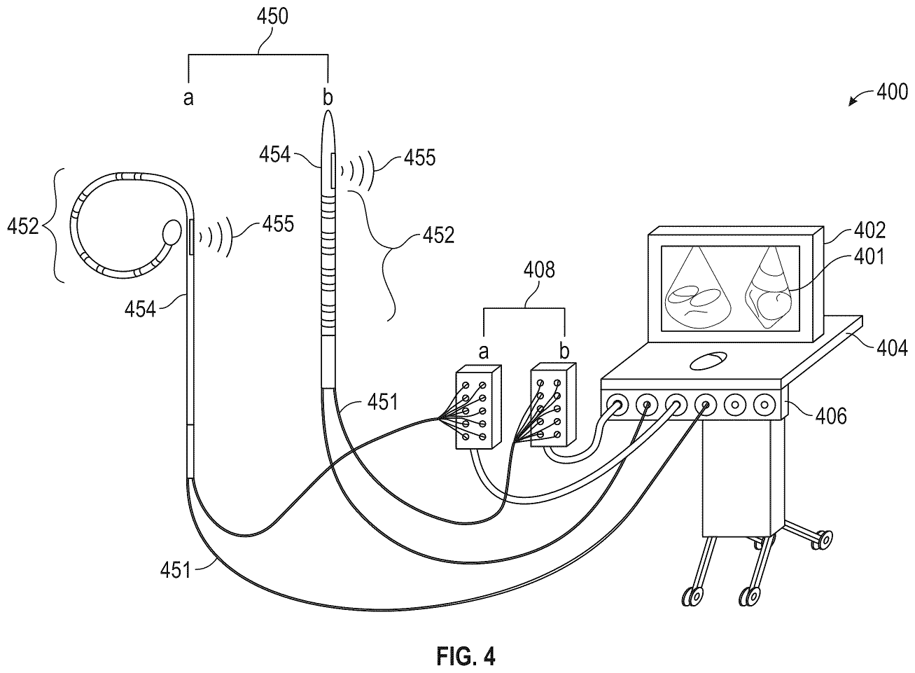

Referring now to FIG. 4 in conjunction with FIGS. 1-3, FIG. 4 illustrates a schematic of imaging console 400, which is an embodiment of imaging console 100 shown in FIG. 1A, wherein similarly labeled parts and numbers correspond to similar features having similar functionality. As shown in FIG. 4, in some embodiments, imaging console 400 includes display device 402 that displays graphic user interface (GUI) 401, computer 404, data ports 406, pin box connector(s) 408a, 408b and catheters 450a and 450b. Catheter 450a is shown as a loop or circular catheter, which may be the same or similar to catheter 250G, discussed above. Catheter 450b is shown as a linear catheter, which may be the same or similar to linear catheters 250A, 250B, 250C, 250D. Other variations of catheters may be implemented as well. In some embodiments, more than two imaging catheters may be utilized by console 400, for example, 3, 4, or more than 4.

In some embodiments, catheter(s) 450a, 450b sense imaging data via transducers 454 and transmit such imaging data via multiple data lines 451 to pin-box connector 408. Pin Box connector 408 advantageously handles each data transmission line in a manner that ensure robust data transmission with negligible signal interference. For example, noise reduction techniques may include shielding and rounding by enhancing the catheter's shielding and providing additional grounding to system 10 to prevent the introduction of electrical noise.

For example, in some embodiments, to enhance the robustness of data transmission and mitigate noise in catheters equipped with one or more transducers, each data line may be individually shielded. This approach involves encasing each data line in a protective conductive layer, which serves to block external electromagnetic interference that could distort the imaging data signals. Shielding is particularly advantageous in medical environments where various electronic devices might operate simultaneously, increasing the potential for signal interference.

In some embodiment, implementing grounding techniques may provide a path for unwanted electrical currents to be safely dissipated into the earth or a grounding system, thereby reducing the risk of electrical noise contaminating the data signals. Thus each line is both shielded and properly grounded. Individual line shielding combined with effective grounding significantly enhances the clarity and reliability of the imaging data transmitted from the transducers to the system, ensuring high-quality diagnostic images and facilitating accurate medical assessments.

In some embodiments, pin boxes 408 may be interchangeable with cables connecting catheter 450 to system 10 directly with all the electrodes' wires bundled in a single cable (e.g., cable 451). Pin-Box connectors 408 may relay signal transmissions to computer 404 via data ports 406. Data ports 406 may be configured for efficient and high speed signal transmission of large amounts of data. In some embodiments, computer 404 includes features for providing interactions with a user (i.e., a clinician).

For example, such features of computer 404 may include display device 402 as, for example, a cathode ray tube (CRT) or a liquid crystal display (LCD) or a light emitting diode (LED) monitor or Augmented Reality (AR) device or Virtual Reality (VR) device worn by a user/physician for displaying information to the user and a keyboard and a pointing device, such as for example a mouse or a trackball (not shown), by which the user may provide input to computer 404. Other kinds of devices can be used to provide for interaction with a user as well. For example, feedback provided to the user can be any form of sensory feedback, such as for example visual feedback, auditory feedback, or tactile feedback; and input from the user may be received in any form, including, but not limited to, acoustic, speech, or tactile input. Other possible input devices include, but are not limited to, touch screens or other touch-sensitive devices such as single or multi-point resistive or capacitive trackpads, voice recognition hardware and software, optical scanners, optical pointers, digital image capture devices and associated interpretation software, and the like.

As mentioned above, some embodiments herein provide integrating multiple real-time image streams from multiple intra-cardiac echocardiogram ultrasound sources. For example, catheters 450a and/or 450b, transmit multiple imaging data streams from multiple transducers 454, which may be reconstructed and rendered on GUI 401. Such reconstruction and rendering provide a comprehensive imaging solution for cardiac diagnostics. Additionally, some embodiments herein facilitate the simultaneous presentation of 2D/3D streaming images from one or more connected ultrasound sources on display device 402, offering a detailed and encompassing view of the cardiac anatomy.

Discussed in detail further below, in some embodiments, 3D and 4D reconstruction may be rendered on display device 402 and further augmented with display of electrical conductivity information via GUI 401. In some embodiments discussed below, system 10 is capable of recording, analyzing and displaying electrical conductivity from body surface ECG leads (e.g., 160) as well as intra-cardiac EGM associated with electrodes 452 touching or in close proximity to cardiac tissue. Discussed in detail further below, the various cardiac electrical related information (e.g., uni-polar voltage, bi-polar voltage, local activation time, low voltage signals, and/or fractionated signals), may be projected on a rendered 2D/3D reconstruction GUI 401 and synchronized with the cardiac cycle to present mechanical motion (i.e., a 4D rendering) of the cardiac muscle with the corresponding cardiac electrical conduction mechanism. In some embodiments, besides the cardiac electrical information, system 10 via console 400 may be capable of projecting and associating additional functional and visual indications of cardiac metrics on the reconstruction as well as the 2D and 3D ultrasound images, which is discussed further below.

For clarity, real-time image streams showing mechanical motion of the cardiac structures are reconstructed from iterative sweeps of high frequency sound waves (i.e., ultrasound) emitted by an ultrasound transducer (e.g.,154, 254, 354, 454), such iterative sweeps are herein referred to as “ultrasound fan”. Multiple iterative sweeps of ultrasounds fans may facilitate the imaging transducer “field of view.” A brief review follows. An “ultrasound fan” refers to the fan-shaped wave pattern that is emitted from an ultrasound transducer during an imaging session. Ultrasound transducers operate by emitting high-frequency sound waves into the body. When such high frequency sound waves encounter different tissues and structures, sound waves are reflected back to the transducer at varying speeds and intensities depending on the density of the tissue the high-frequency sound waves hit.

For example, transducer 154, 254, 354, 454 (hereinafter, collectively “transducer(s) 454) acts both as a speaker (to send ultrasound waves) and a microphone (to receive ultrasound waves). The returning ultrasound echoes are captured by the transducer and translated into electrical signals. Such electrical signals are then processed by the ultrasound machine (e.g., server 102 and/or console 100, 400) to create real-time images of cardiac anatomical structures or internal structures of the heart, such as chambers, valves, and blood flow. Doing so provides for detailed examination of the cardiac features, enabling the detection and assessment of conditions like valve abnormalities, wall motion, and other cardiac anomalies. Accordingly, as utilized herein a “fan” or “field of view” refers to the sweeping motion of an ultrasound beam 455 emitted by transducer 450, as ultrasound beam 455 moves across an area of interest, such as heart 32. Ultrasound beam 455 is directed outwards in a fan-like shape from the transducer, which allows for a broad area to be examined. The term “fans” may also refer to the sequential slices or image planes that are acquired as ultrasound beam 455 sweeps through the tissue of heart 32. For example, for a 2D image, multiple “fans” or sweeps are collected in a single plane to create a flat, two-dimensional view of the heart. These can be seen in real-time, providing a moving image that can show heart function, blood flow, and valve movement. For 3D imaging, the ultrasound system collects multiple “fans” across multiple planes.

Accordingly, in some embodiments, rendering module 112 reconstructs such planes into a three-dimensional image of the heart for display on GUI 401. Doing so may advantageously provide a more comprehensive view of the heart's anatomy and function, and is advantageous for understanding complex structures and relationships within the heart that are not easily observed on 2D imaging. The number of “fans” collected and the resolution of the image will vary based on the specific ultrasound transducer used and the type of study or procedure being performed.

Connecting with multiple ICE ultrasound sources, (e.g., catheters 450a, 450b) advantageously enhances the scope and depth of cardiac imaging. In some embodiments, system 10 employs techniques for improving the accuracy of precise localization of such ultrasound sources provided by catheters 150, 250, 350, 450 (hereinafter collectively “catheter(s) 450”). For example, in some embodiments, such functionality is provided by via magnetic sensors (e.g., 353) placed in close proximity to one or more transducers 454, shape-sensing fiber optic wires (e.g., flex circuit 362), and/or other advantageous localization methods, discussed in further detail below, thereby ensuring accurate determination of the position and orientation of each, some or all, transducers in catheter(s) 450.

Some embodiments described herein provide the capability of reconstructing 3D images of cardiac anatomy. In some embodiments, system 10 advantageously identifies anatomical structures via user selected, or predictive model, delineation of structures. Identifying anatomical structures enables users to accurately mark and analyze cardiac feature contours on rendered ultrasound images and streams. Such contour labeling is advantageous for detailed structural analysis and increases accuracy of diagnosis.

In some embodiments, system 10 is uniquely designed to support both static and dynamic anatomical reconstructions. The static reconstruction provides a detailed view of the anatomy at specific phases of the cardiac cycle, while the dynamic reconstruction offers a real-time depiction of cardiac wall movements throughout the cycle. This dynamic feature is advantageous in understanding the intricate functions and morphology of the heart, which is discussed in further detail below. Rendered 3D/4D images may be displayed on system 10 display device 402, offering a vivid and dynamic representation of the heart, which is described in further detail below.

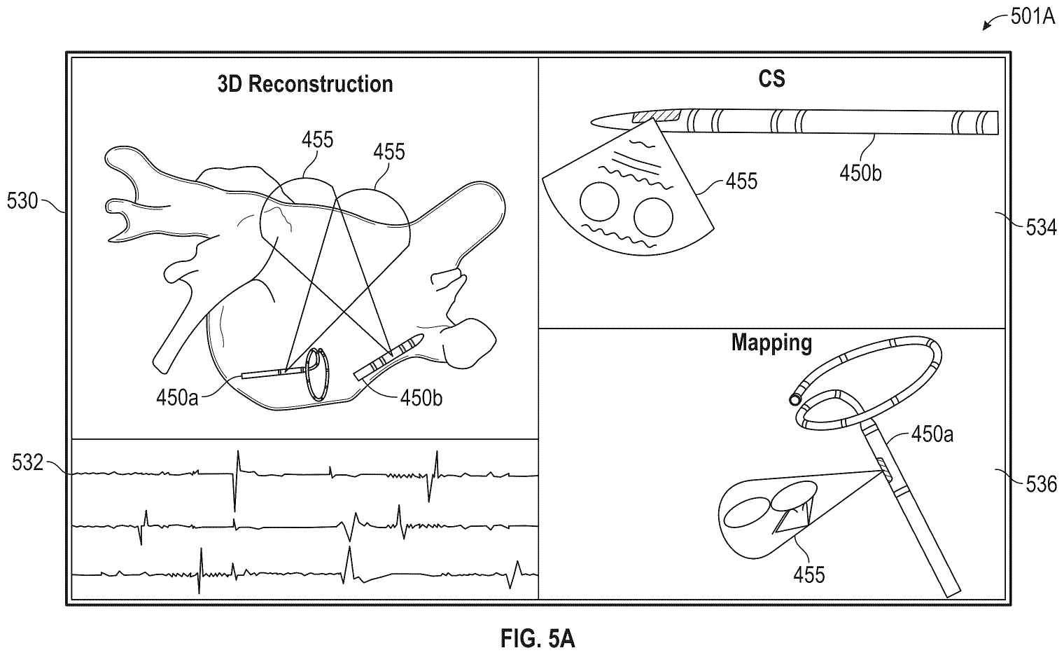

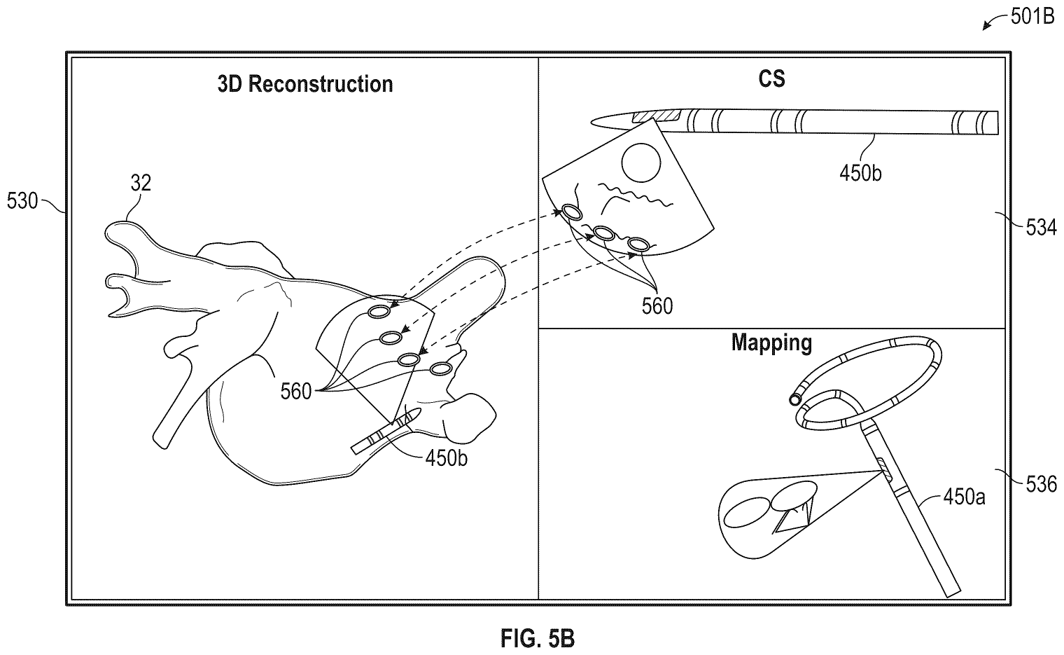

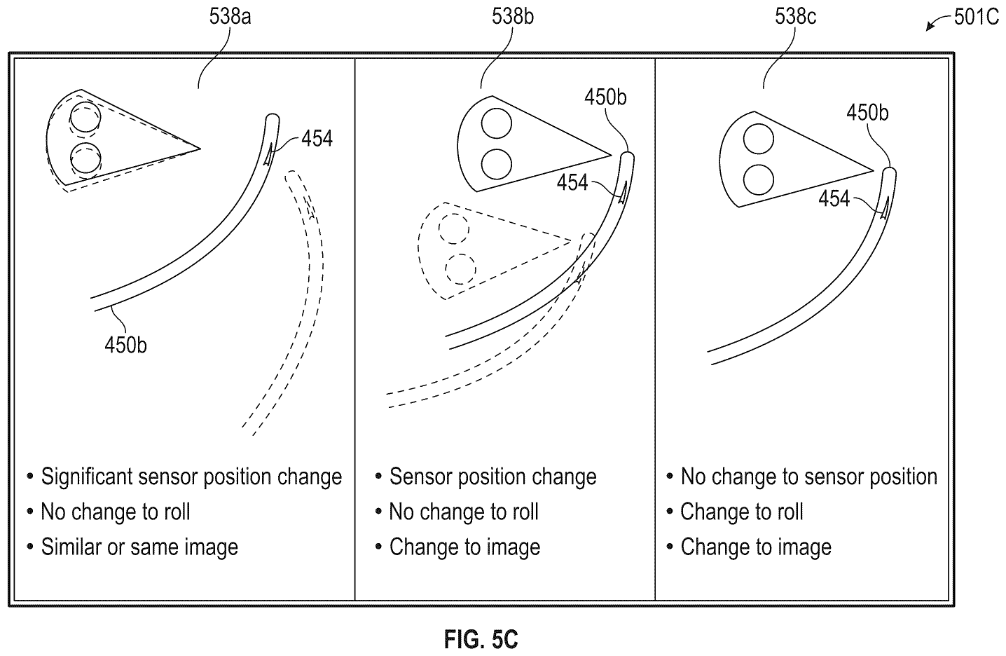

Referring now to FIGS. 5A-5C in conjunction with FIG. 1-4, FIGS. 5A-5C depict embodiments of a graphic user interface 501 for use in console 100, 400. GUI 501 is an embodiment of GUI 401, with similar features and functionality. As shown in FIG. 5A, in some embodiments, GUI 501 may include a real-time reconstruction 530, vital monitoring 532, and imaging catheter displays 534, 536 showing imaging information from multiple catheter(s) 450.

As mentioned above, system 10 is operable to implement a method of combining real-time streams of images from multiple ultrasound sources. The method includes connecting to multiple intra-cardiac echocardiogram ultrasound sources to simultaneously present 2D and 3D, static and streaming images, displayed via multiple tiles or screens on GUI 501. In some embodiments, the method includes localizing the ultrasound sources position and orientation, utilizing 6DOF magnetic sensors (e.g., 353) coupled to transducer(s) (e.g., 354), and identifying anatomical structures, either selected by the user or predictively determined from multiple catheters (e.g., 350). The method includes reconstructing the cardiac chambers from the ultrasound sources' images, and providing 2D and 3D view of the cardiac anatomy and full chamber real-time reconstruction (e.g., 530), which is discussed in further detail below.

### Localization Tracking

In some embodiments, system 10 is capable of rendering catheters, sheaths or other devices and objects used during cardiac treatment by using localization techniques such as 6DOF tracking, impedance-based localization, and/or via shape sensing fiber optics. Such rendering and applied localization techniques may improve the user's ability for positioning transducers relative to any cardiac anatomy reconstruction, in the same 2D or 3D view, which is discussed further below. Objects localized by system 10 as well as the anatomical reconstruction of heart 32 are tracked and synchronized among system 10 components (108, 110, 112, 114, 116) to a global coordinate system (discussed further below), which, in some embodiments, may be managed by coordinate sync module 110.

Tracking 6DOF by way of magnetic sensors refers to the ability to determine an object's precise position and orientation in three-dimensional space. As utilized herein, DOF, or degrees of freedom refers to the number of independent movements an object may make. For example, 5DOF includes three translational movements (e.g., up/down, left/right, forward/backward) and two rotational movements (e.g., pitch and yaw). And 6DOF adds another more rotational movement (e.g., roll). In some embodiments, one or more of electrodes 152, 252, 352, 452 (hereinafter, collectively “electrode(s) 452) may include, or may be coupled to, a 5DOF or 6DOF magnetic sensor 353, which provides advantageous spatial data. Such spatial data may be processed under 5DOF or 6DOF protocols, thereby facilitating precise localization of the catheter and catheter components (e.g., electrode(s) 452 and/or transducer(s) 454), which is discussed in detail below.

For example, referring back to FIG. 3, in some embodiments, one or more 6DOF magnetic sensors 353 may be: coupled to transducer(s) 454, placed on shaft 257, 357, 457, and/or embedded with or placed near electrode(s) 452. Such 6DOF magnetic sensors 353 output spatial data, which is analyzed to localize and track tip 358 and the orientation of the transducer(s) 354 field of view within heart 32. For example, 5DOF tracking may include information on an electrode's location in three-dimensional space (x, y, and z coordinates) and two rotational axes. In contrast, 6DOF includes an additional rotational axis, providing complete spatial orientation—this includes pitch, yaw, and roll. Such tracking is advantageous for navigating catheters(s) 450 through complex anatomical pathways, such as those in heart 32, during minimally invasive procedures.

In some embodiments, under a 5DOF protocol, system 10 may determine catheter(s) 450 position and orientation in three-dimensional space and along two rotational axes, respectively. In some embodiments, 6DOF magnetic sensor data includes and facilitates determination of 6DOF localization parameters (e.g., up/down, left/right, forward/backward, pitch, yaw, roll), allowing system 10 to understand how catheter(s) 450 is positioned and angled inside heart 32. Advantageously, 6DOF protocol, which extends 5DOF by adding the ability to detect or determine roll (i.e., rotation around the catheter's longitudinal axis), gives a more comprehensive understanding of the catheter's orientation, which is advantageous for complex navigation within the heart's chambers and vessels.

In some embodiments, system 10 may include electrode-based positioning functionality. As mentioned above, catheter(s) 450 may be equipped with multiple electrode(s) 452 distributed along the length of the catheter shaft. Such electrodes are not only for sensing electrical activity within the heart but also serve as markers for localization via impedance measurement. Accordingly, in some embodiments, system 10 may determine transducer(s) 454 localization via analyzing impedance data from the electrode(s) 454. Such impedance data may inform system 10 about a specific segment of catheter(s) 450 and thus transducer(s) 454's relative position and orientation. Because the orientation and position of the transducer affect the imaging field of view 355, 455 quality, such localization determinations are advantageous for increasing the accuracy of 3D image renderings.

For example, by measuring impedance (the resistance to electrical current) at different points along catheter(s) 450, system 10 may determine the catheter's shape and curvature within heart 32. Because impedance varies with tissue contact and proximity, such impedance measurement may advantageously provide spatial information. In some embodiments, electrodes 352 transmit impedance data back to a processing unit (e.g., 114). Such impedance data reflects the interaction between catheter(s) 450, and cardiac tissue, as well as the catheter's orientation and position.

Referring to FIG. 5B, FIG. 5B shows GUI 501B, displaying markers 560, which are shown in screen 530, showing a dynamic reconstruction of the cardiac structure having markers 560 shown on screens 530, 534. In some embodiments, markers 560 may aid when locating and orienting catheter(s) 450 for targeted treatments, such as ablation or stent placement. Marking objects, sensitive areas like scars, ablation markers, and surgical devices like pacemakers may be advantageous when needing to avoid sensitive areas. Because the localization techniques implemented in the embodiments herein provide a highly accurate means to track the shape and position of a objects and devices in real time, such localization techniques advantageously may provide benefits in terms of precision and reliability for complex medical procedures.

In some embodiments, system 10 transfers and associates anatomical structures identified in the ultrasound image to the 3D reconstruction of the cardiac chambers and vice versa. For example, in some embodiments, identifying the coronary sinus ostium may include, the user placing a marker (e.g., 560) on the 2D or 3D ultrasound view (e.g., 530) and such marker is visible in the 3D reconstruction view 530 so that a localized linear catheter may aim for that marker to be placed inside the CS vein.

In some embodiments, system 10 may connect to ablation catheters and localize and track the position of such ablation catheters. System 10 via console 100, 400 may detect when ablation is performed, either by receiving an indication from the ablation generators or selected by the user. When ablation is performed, system 10 records the ablation locations and projects the ablation markers (e.g., 560), on a static display of the reconstruction, on dynamic display of the reconstruction, and/or on the 2D and 3D ultrasound real-time image.

In some embodiments, system 10 is capable of dynamic adjustments to localization tracking. For example, as catheter 250 moves, the patient moves, or the heart contracts, electrodes 252 along shaft 257 continuously provide updated impedance data, allowing system 10 to dynamically adjust catheter transducer 254's perceived position, ensuring accurate, real-time imaging, which is discussed in further detail below.

For example, as shown in FIG. 5C system 10, utilizing spatial data, is operable to detect and display alert(s) when catheter(s) 450 moves in the coronary sinus vein due to the patient moving or catheter slipping, and/or catheter being adjusted by the clinician. For example, system 10 may detect catheter 450b and utilize the catheter ultrasound image or magnetic sensor information or both to detect and display a visual indication of when the patient moves (as shown by 538a), the catheter slipping in the CS vein without the patient moving (as shown by 538b), and/or the adjustment of ultrasound transducer orientation without the patient moving (as shown by 538C).

For example, when system 10 detects significant position changes of the magnetic sensor with no change to the ultrasound image, system 10 determines that the patient has moved. In some embodiments, when system 10 determines that significant position change of the sensor with no significant change to the magnetic sensor roll and significant change to the ultrasound image, system 10 determines that the CS catheter has slipped in the CS vein without the patient moving. In some embodiments, when system 10 determines no significant change of the magnetic sensor, significant change to the magnetic sensor roll, and significant change to the ultrasound image, system 10 determines an adjustment of the transducer orientation has occurred.

Referring back to FIG. 1, in some embodiments, localization module 108 and coordinate sync module 110 work in tandem to implement 6DOF/5DOF tracking in catheter(s) 450 and/or other devices and objects in heart 32. Localization module 110 may utilize 6DOF magnetic sensors, flex circuit 362, and/or electrode(s) 452, to determine the precise position and orientation of the catheter in three-dimensional space. Meanwhile, coordinate sync module 110 and object tracking module 114 integrates such tracking data into a global spatial framework. Such global coordination ensures that the catheter's movements are accurately represented and synchronized with the system's 3D model, facilitating precise navigation and positioning within the patient's heart. For example, in some embodiments, rendering module 112 may render a mapping of such global coordinate system for display in real time on display 402, which is discussed in detail further below.

In some embodiments, system 10, via object tracking module 114, may identify and store positioning information (e.g., 6DOF tracking) for objects and devices placed in heart 32 when such object position intersects with the ultrasound image, as shown by display 536. Identification of objects and devices (e.g., electrodes, transducers, catheters, medical devices, and cardiac structures) that appear in the ultrasound image may be selected by the clinician or predictively identified based on the object or device's shape using image processing techniques that may be aided by localization information or spatial data in instances where the object or device is being localized by localization module 108.

In some embodiments, predictive module 116 may recognize objects and devices based on their unique or known shapes. For example, shape recognition may be implemented using image processing algorithms that analyze ultrasound images and recognize objects and devices based on a unique shape of such objects and devices. For example, in some embodiments, image processing algorithms may detect specific geometric patterns, contours, and sizes that correspond to known medical devices such as phase array ultrasound transducers, electrodes, and other catheter components. For example, in some embodiments, system 10 may assess the contrast and texture within the ultrasound images for determining a position and location of a device. Because medical devices often have different acoustic properties compared to biological tissues, such acoustic properties may be exploited to distinguish non-biological tissues in the image. In some embodiments, resources 120 may include additional predictive models, which are trained on large datasets of ultrasound images containing various medical devices. These models may learn to identify and differentiate devices based on a unique shape and appearance in the ultrasound scans.

In some embodiments, system 10 may determine the spatial relationship between objects and devices and anatomical structures or cardiac tissues rendered in GUI 501.

For example, system 10 may determine and indicating the proximity and contact between objects, devices and tissue. System 10 may also monitoring whether catheters are applying consistent or inconsistent pressure on cardiac tissue during heartbeats. In some embodiments, system 10 may determine and/or present a rendering to the user of the distance between a catheter or device and one or more anatomical structure(s) or cardiac walls and render such determinations and calculations on a GUI of system 10, which is discussed in detail further below.

Referring back now to FIG. 5C, in some embodiments, system 10 uses a catheter ultrasound image sensor coupled to 6DOF magnetic sensors for detecting and provide visual indication of when a patient moves. For example, when significant position changes of the magnetic sensor are sensed and no corresponding change to the ultrasound image. In some embodiments system 10 may determine that the CS catheter slip in the CS vein without the patient moving and render an alert to the user/physician. For example, when a significant position changes of the magnetic sensor and no significant change to the magnetic sensor roll but significant change to the ultrasound image.

In some embodiments, system 10 may detect an adjustment of the ultrasound transducer orientation. Such detection may include sensing no significant change to the magnetic sensor position and significant change to the magnetic sensor roll, with significant change to the ultrasound image.

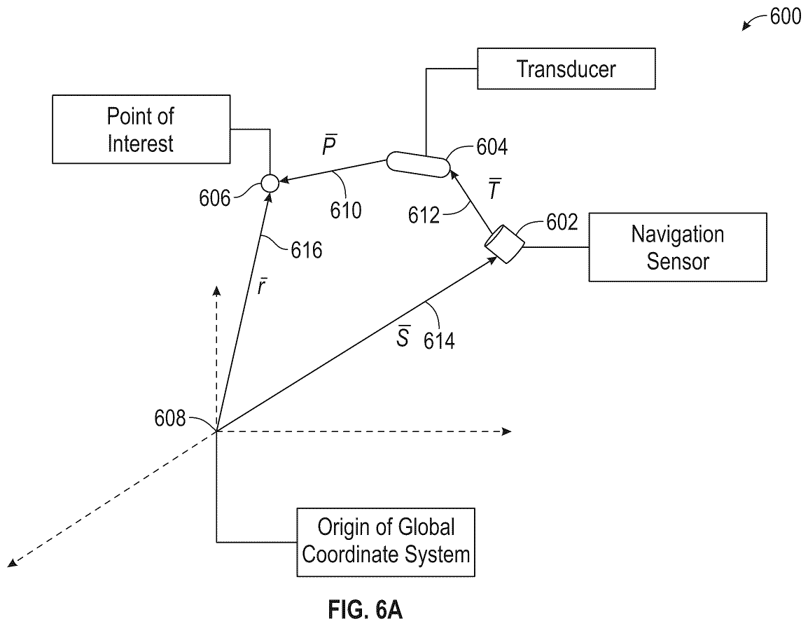

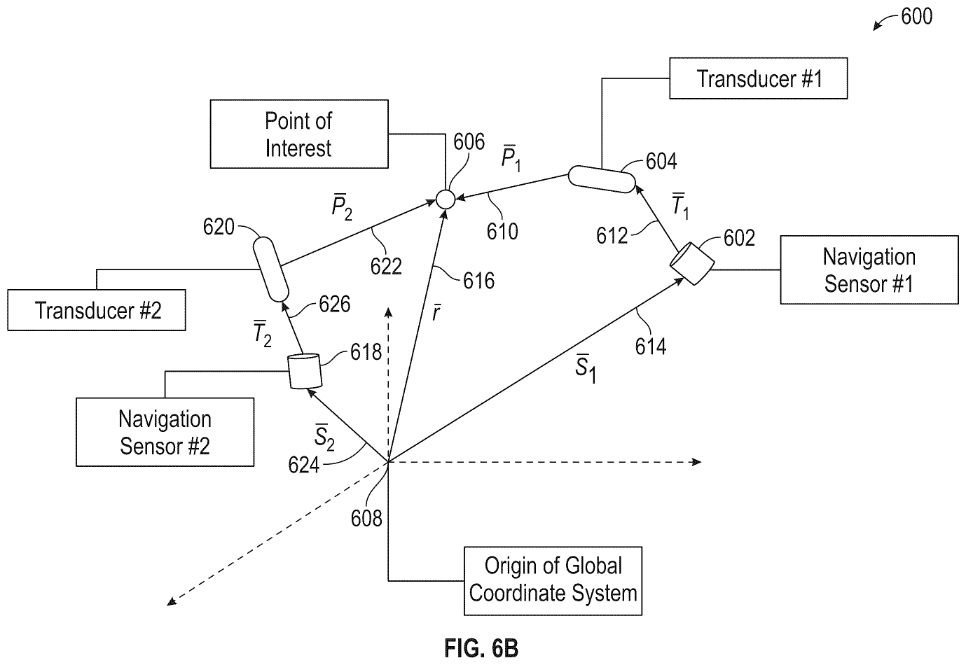

Referring now to FIGS. 6A-6B, FIG. 6A-6B depicts of a global coordinate map 600 shown as a 3D spatial mapping of catheter components in a 3-dimensional Cartesian coordinate plane. For 3D coordinate systems in medical imaging of transducers in intracardiac procedures, the position and orientation of a transducer is advantageous for accurate imaging and navigation. Accordingly, in some embodiments, coordinate synchronization module 110 may manage global coordinate map 600. As shown, map 600 may include navigation sensor 602, 618 transducer 604, 620, point of interest 606, and global origin 608. Global origin 608 serves as the reference for all measurements and is the fixed point from which vectors (e.g., 612, 614, 616, 620, 622, 624, or 626) are determined. Global origin 608 may be a predefined location within the heart or an external reference point, for example, a voltage reference electrode.

The position of transducer 604 in the 3D space is advantageous for targeting and obtaining clear images of point of interest 606. Accordingly, navigation sensor 602, 618 may provide real-time data on the location and orientation of transducer 604. Navigation sensor 602, 618 may be advantageous for guiding transducer 604, 620 to the correct position within the heart, for example, point of interest 606. Point of interest 606 may correspond to a target location that needs to be imaged or treated, such as an area of cardiac tissue within the heart. Transducer 604, 620 must be advantageously oriented to acquire the best possible image of point 606. Such orientation may be determined via vector analysis, discussed further below.

For example, vectors include position vector (P) 610, transducer vector (T) 612, and sensor vector (S) 614. Position Vector (P) 608 represents the position of the point of interest relative to global origin 608. P 608 provides the spatial coordinates needed to locate point of interest 606 in 3D space. Transducer Vector (T) 610 may indicate the orientation or the positional relation of transducer 604 with respect to navigation sensor 602, or another reference point (e.g., point of interest 606). Sensor Vector (S) may represent the orientation or position of navigation sensor 602 within global coordinate map 600.

Some embodiments herein provide improving accuracy of a given point on interest in the 3D coordinate system using multiple transducers. For example, in some embodiments, catheter(s) 450 with transducer(s) 454 also include navigation functionality. For example, the location of a given point of interest 606 that is visualized by transducer 604 may be described by the sum of three vectors:

$$\overline{r} = \overline{S} + \overline{T} + \overline{P} \qquad ( Eqn . 1 )$$

Where r̅ is the location of a point of interest 606 in global coordinate map 600, S̅ is the location of navigation sensor 602, T̅ is the location of transducer 604, and P̅ is the location of point of interest 606. The values of each of the vectors S̅, T̅, P̅ has an inherent uncertainty, which results in some uncertainty in the value of r̅.

Accordingly, in regions where the “field of view” of two or more transducers overlap, as shown in FIG. 6B, the uncertainty in the value of T may be attenuated by implementing the following feature:

$$\overline{r} = \overline{{S}_{1}} + \overline{{T}_{1}} + \overline{{P}_{1}} = \overline{{S}_{2}} + \overline{{T}_{2}} + \overline{{P}_{2}} = \overline{… = {S}_{ι}} + \overline{{T}_{ι}} + \overline{{P}_{ι}} \qquad ( Eqn . 2 )$$

Eqn. 2 yields a more accurate image and location mapping when compared to Eqn. 1 alone. In Eqn. 2, the subscripts indicate the different transducers. The uncertainty in the location of each point may be advantageously scaled as, \sqrt{N}/1, where N is the number of transducers that “see” that point.

Thus, improving the image presented to the clinician may be achieved by enhanced location mapping as discussed above. As each of the transducers 604, 620 provides an image, the location of several shared key points in those images may be improved via vector analysis as described above. Such points of interest with enhanced position accuracy may be used as fiducial points. Once fiducial points are ascertained, such fiducial points may be used to register the entire or substantially the entirety of an image provided by each transducer-including the part(s) of the image that resides outside of the region(s) of overlap (i.e., regions outside of the intersection of two or more transducer fans). Such functionality improves the overall image presented to the clinician.

In some embodiments, selection of fiducial points may be selected by the clinician. In other embodiments, fiducial points may be determined by predictive module 116 and/or resources 120. Such selection would rely on selected image metrics, which may include one or more image features, and/or cardia features. In some embodiments, image features may include, for example, resolution, signal-to-noise-ratio, texture analysis, edge detection, and/or depth perception. In some embodiments, cardia features may include physiological features such as the intra-atrial septum, mitral, tricuspid, pulmonic and aortic valves apparatus, left atrial appendage, pulmonary veins ostia, coronary sinus, vein of marshal, superior vena cava, right atrial appendage. Cardiac features may also include features of catheter(s) 450-such as electrodes whose relative distance from each other is known a-priori.

Image metrics are advantageous in evaluating the quality and effectiveness of the images beyond just contrast features and motion. The resolution of an image is advantageous as it determines the sharpness and clarity, thereby facilitating fine granularity. Signal-to-Noise Ratio (SNR) assesses the strength of the signal in comparison to the background noise and advantageously influences the overall clarity of the image. Texture analysis may be employed for evaluating variations in brightness or color within an image, a key factor in identifying specific tissues or abnormalities. Edge detection advantageously enables the precise identification of the borders of objects in an image, essential for accurately delineating different structures. And depth perception becomes advantageous for the accurate perception of the depth and positioning of structures within the body. In some embodiments, such image metrics may be utilized by rendering module 112 for displaying real-time 3D renderings and collectively by server 102 modules (e.g., 108, 110, 112, 114, and 116) for ensuring the accuracy and reliability of system 10 imaging data.

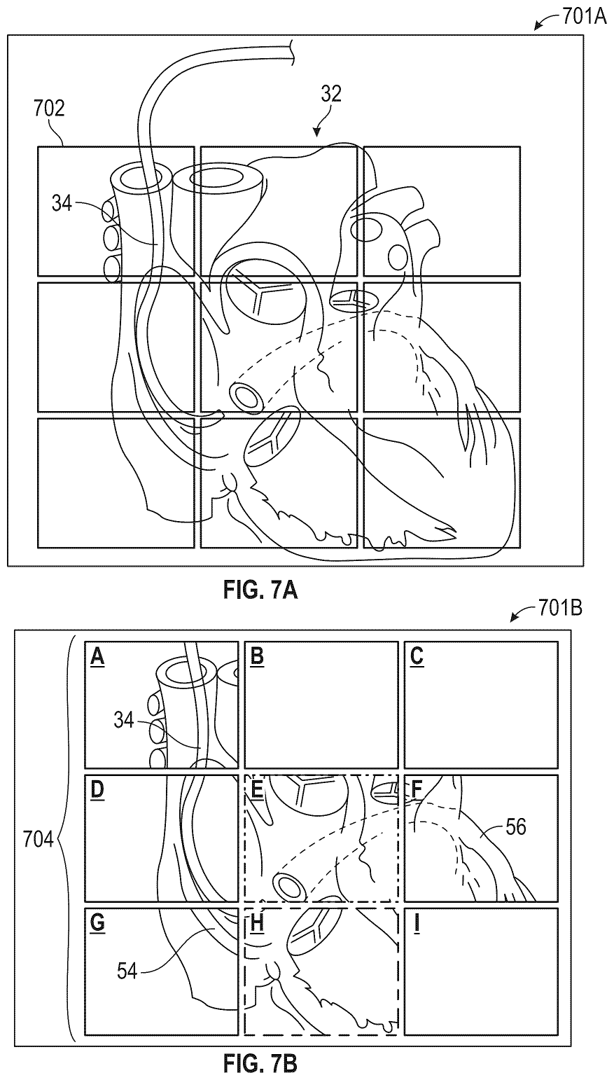

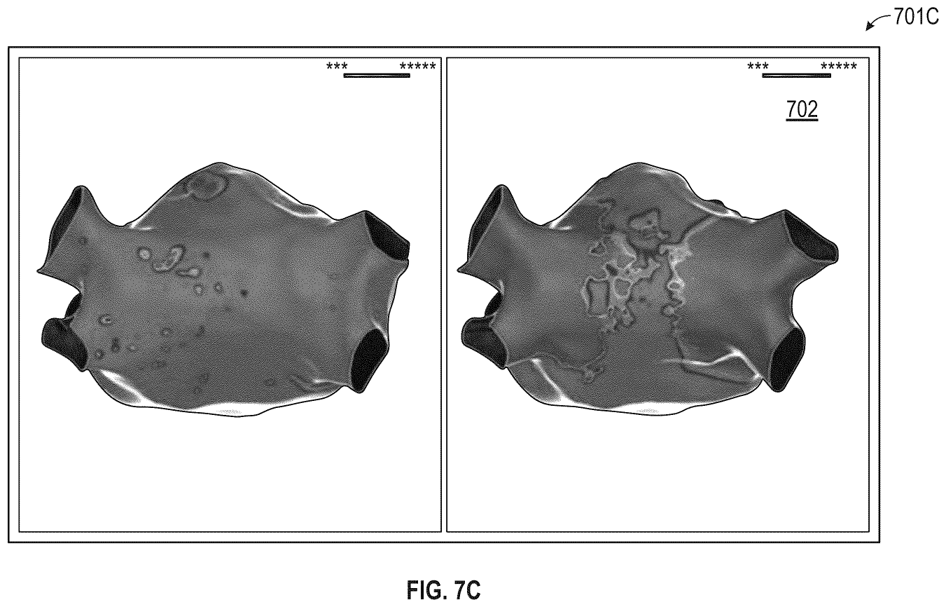

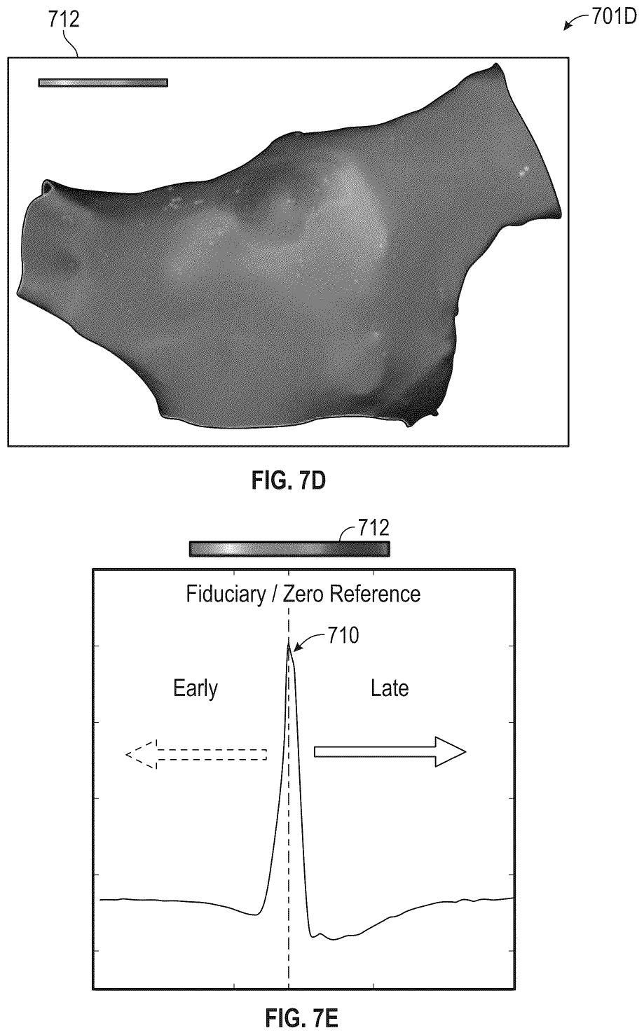

Referring now to FIGS. 7A-7E, in conjunction with FIGS. 1-6, FIG. 7A depicts an exemplary GUI 701 for displaying 2D and 3D renderings of ultrasound imaging data. GUI 701 is configured for use in console 100, 400. GUI 701 is an embodiment of GUI 401, 501 wherein similarly labeled parts and numbers correspond to similar features with similar functionality. In some embodiments, GUI 701 includes a graphical representation of a heart having overlay 702, overlaid with a grid, which divides the rendering into tiles 704A-704I. Such visualization is advantageous in medical imaging software to facilitate more precise navigation and manipulation within a 3D space.

For example, by segmenting heart 32 into tiles 704A-704I, system 10 may allow clinicians to focus on specific areas for detailed analysis, interventional planning, or real-time monitoring during procedures. Some, at least one, or each tile 701a-701i may represent a volume of interest where specific diagnostic or therapeutic actions may be targeted, such as in ablation therapy where accurate localization is advantageous. Overlay 702 may aid in aligning the multiple data points, or fiducial points, collected from different imaging transducers 604, 620, advantageously enhancing the overall accuracy of cardiac procedures.

In some embodiments, system 10 is capable of continuously associating positions of objects and devices in global coordinate map 600 and use such positions to contribute to the cardiac anatomy reconstruction shown in GUI 701. Since trackable objects are localized on global coordinate map 600, the positions (e.g., point of interest 606) sampled may be used to identify areas that are categorized as either placed in the blood pool, or touching anatomical structures, like cardiac wall tissue. Such position tracking contributes to the cardiac anatomy reconstruction regardless of the reconstruction originating partly from various ultrasound sources or from various catheters in a multi catheter implementation.

As shown, GUI 701A may display reconstructed transducer imaging data into a 3D/4D images, providing multiple, potentially overlapping views of anatomical structures. Tiles 704a-704i in GUI 700 may function as a distinct “window,” offering a unique view of various parts of the heart's anatomy. For example, in some embodiments, rendering module 100 and/or system 10 may program tiles 704 to focus on specific, different, or the same anatomical regions, with the option to have multiple tiles 704 observing the same structure for broader surveillance. Tile 701 may be rotated to display the same anatomical structure from a different perspective. Such tile rotation functionality is advantageous for comprehensive cardiac examinations, and facilitates users to view areas of interest from multiple angles without repositioning the transducer manually.

In some embodiments, the number of tiles 704 on GUI 700 may include more or less than what is shown in FIG. 7, thereby providing flexibility in monitoring. Adjusting the number of tiles may be advantageous in complex procedures where attention needs to be divided among various critical areas. The size of the tiles can also be manually adjusted, allowing for more detailed visualization of specific structures or a broader view when necessary. For example, tile size adjustment functionality provides clinicians with the flexibility to enhance or reduce the scale of the visual field for each tile 704. During a procedure like cardiac ablation, a larger tile may display an expanded view of the atrium to assess overall anatomy and the locations of interest, such as the pulmonary veins. Conversely, a smaller tile may focus on a detailed area to closely observe the tissue's response to ablation. Such adjustability is advantageous when precision is imperative, such as verifying the ablation points' alignment with the targeted arrhythmic pathway.

In some embodiments, GUI 700 includes displaying tile state data for tiles 704. The tile state functionality within the GUI of a 3D imaging transducer system 10 adds a dynamic layer to cardiac imaging, particularly in procedures like ablation. In some embodiments, rendering module 112 may display tile states via GUI 700 corresponding to a real time state, static state, and/or initial state.

In some embodiments, real time-state include tile 700 actively displays live images from a transducer currently oriented such that its ultrasound fan intersects the assigned area (e.g., point of interest 606). Such real-time imaging is advantageous during dynamic procedures, allowing clinicians to see the effects of their actions, like ablation, as such effects happen. A static state of tile 704 is enabled when the transducer has moved away from a previously imaged area, such static state tile assigned to previously imaged area may hold a “frozen” image. Such frozen image may be last captured image before the transducer's orientation changed, which is advantageous for comparison with live images of the same point of interest of cardiac structure, to check progress over time, or to maintain a visual reference of a critical area during the procedure.

In some embodiments, an initial state of tile 704 may occur when a tile is not actively displaying live or static images. Such initial state may show an empty space or a standard anatomical model relevant to the tile's designated region. The standard model may include a generic image of the heart or a specific part thereof, serving as a placeholder or a guide for what the clinician may expect to see. Such initial state of tile 704 is helpful for planning the procedure or when awaiting the initiation of live imaging.

Accordingly the so-called tile states implemented in GUI 701 in accordance with some embodiments, enhances system 10's utility, offering a tailored approach to visualizing cardiac anatomy and the ablation process, and enabling clinicians to switch between historical and live data views as needed for optimal patient care. Such tile states may be displayed by color coding the frame of each tile or providing a different border outline for each individual tile state (e.g., tile 704e) Thus, the integration of GUI 701 in a 3D imaging transducer system enhances the functionality of cardiac imaging, providing a sophisticated platform for real-time monitoring, diagnosis, and potentially guiding interventional procedures. Such tiled approach offers a customizable and dynamic user experience, adapting to the varying demands of different cardiac treatments.

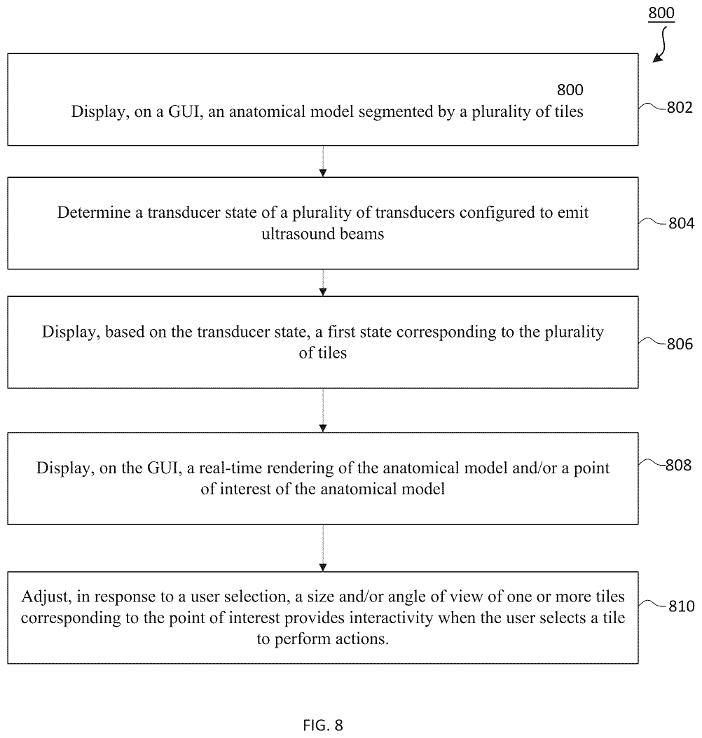

For example, referring to FIG. 8, FIG. 8 depicts a flow chart for an exemplary clinical process, real-time clinical flow 800. Clinical flow 800 may include use of 3D imaging transducer system 10. At operation 802, clinical flow 800 includes displaying, on a GUI, an anatomical model segmented by a plurality of tiles. Providing a number of empty tiles 704 on GUI 700 is shown similar to tile B, for example, akin to a blank canvas. In some embodiments, tiles 704 may be pre-populated with an average anatomical model, effectively dividing heart 32's anatomy across tiles 704 for initial planning and orientation. At an operation 804, clinical flow 800 includes determining a transducer state of a plurality of transducers configured to emit ultrasound beams, and at operation 806, displaying, based on the transducer state, a first state corresponding to tiles 704. Thus, as the transducers become active, corresponding tiles transition into a real-time state (e.g., tile 704e, 705h), and the GUI may display live imaging where the ultrasound “fan” or beam intersects the designated anatomical area or point of interest. Such live image display advantageously allows clinicians to view the anatomy and any interventions in progress.

In some embodiments, clinical flow 800 includes, at operation 808, displaying, on the GUI, a real-time rendering of the anatomical model and/or a point of interest of the anatomical model. For example, in some embodiments, system 10 may determine when one or more ultrasound fan is no longer intersecting an area or point of interest, the relevant tiles 704 shift to a static state (e.g., tiles 704a, 704d, 704f, and 704g), preserving the last captured image. Such historical view may be advantageous for tracking changes over time or maintaining situational awareness during cardiac procedures. At an operation 810, in response to a user selection, adjusting a size and/or angle of view of one or more tiles corresponding to the point of interest provides interactivity when the user selects a tile to perform actions. At such point the tile becomes “active” (demonstrated by tile 704e). In such active state, the user may manipulate tiles 704, such actions may include for example resizing or rotating the view for better visualization, or using GUI tools 702 within the GUI to measure distances or perform other analytical tasks.

Throughout clinical flow 800, server 102 modules, including the rendering, 3D coordinate, and localization modules, work in concert. The rendering module updates the visual display; the 3D coordinate module maintains spatial consistency across the tiles; and the localization module ensures that the position and orientation of the ultrasound fans are accurately reflected in the real-time and static images. During a cardiac procedure for example, the localization module tracks the position of the transducers as they move. The 3D coordinate module uses this information to adjust the virtual camera angle in the rendered image, allowing each tile on the display device's GUI 700 to show different views of the same anatomical structure without manual repositioning of the transducers.

As shown in FIG. 7C, for example, in some embodiments, GUI 701 is configured for a catheter with multiple transducers, and the tools for measuring distances and performing other analytical tasks in real-time. For example, GUI tools 702 may include one or more of: a distance measurement tool (allows clinicians to measure distances between two points in real-time, such as the size of a lesion or the thickness of myocardial tissue), volume estimation tool (for procedures requiring volumetric analysis, like assessing the size of a cardiac chamber or the volume of an ablated area), a pathway tracking tool (tracks the path of the catheter through the heart, helping clinicians to ensure that they cover all areas of interest during the procedure), and/or 3D Reconstruction Dual transducer 3D reconstruction tool (offers more complete 3D reconstructions by filling in gaps that might be missed by a single transducer, providing a richer and more detailed image of the cardiac anatomy). Some embodiments include a comparative analysis tool that overlays or juxtaposes images from the two transducers for a comparative analysis of pre-and post-procedure states or different angles of the same area. Such comparative analysis and reconstruction via the dual transducer setup, leverages the multiple perspectives and richer data provided to deliver a more nuanced view of the cardiac environment, facilitating precision in diagnostic and therapeutic interventions. For example, while monitoring the progress of a stent placement, the system can display a frontal view of the coronary artery in one tile, while another tile may show a cross-sectional view from the side, both updated in real-time as the procedure progresses. Such multi-angle capability ensures that clinicians have a comprehensive view of the procedure, contributing to improved outcomes and patient safety.

Referring back to FIG. 1, in the context of cardiac ablation procedures, where precise targeting of tissue is crucial, the functionality of system 10 may significantly enhance the intervention's efficacy. For example, during atrial fibrillation ablation, rendering module 112 may visualize the pulmonary veins' entrance to the left atrium in tile 701A, while tile 701B may simultaneously provide a close-up view of the ablation catheter's tip to monitor tissue contact and lesion formation. 3D coordinate sync module 110 ensures that both views are accurately oriented within the heart's anatomy, while localization module 108 tracks the catheter's position, allowing the clinician to rotate the view in any tile to assess the lesion's depth and circumference, all without physically moving the catheter. This multi-perspective visualization aids in confirming complete circumferential scars are created around the pulmonary veins, a critical factor in the procedure's success.

In some embodiments, system 10, by having a minimal voltage threshold, identifies areas where scarred cardiac tissue is located based on either ultrasound images, device positions or an interpolation of both having a voltage lower than the threshold. System 10 may augment voltage maps, Local Activation Time (LAT) maps or anatomical reconstruction with visual indication of such areas. Scars may be projected on: static reconstruction, dynamic reconstruction, and/or 2D or 3D ultrasound image, via GUI 701. In some embodiments, system 10 supports determining and displaying cardiac metrics indicating wall motion ranging from no motion to full motion and may augment such metric data, numerically and as a color/gray scale, on other information (e.g., voltage, LAT, scar tissue, and the like). In some embodiments, cardiac wall motion metrics may be projected on: static reconstruction, dynamic reconstruction, and/or 2D or 3D ultrasound images.

In some embodiments, system 10 can indicate proximity and actual touch between devices and cardiac tissue when both are observed in ultrasound image. When a device is designed to touch large cardiac tissue area, system 10 indicates full, partial or no contact appropriately. For example, Left Atrial Appendage Occlusion (LAAO) device may fully cover the appendage, or have a sub-optimal placement with parts of the device not being in contact with the tissue. A circular ablation catheter for Pulmonary vein isolation (PVI) may not be in proper contact with the vein ostium and additional positioning and ablation may be required to properly isolate the vein. In some embodiments, based on real-time ultrasound 2D and 3D images system 10 is capable of informing the physician on heart conditions, for example, by identifying a perforation or other cardiac injury.

In some embodiments, system 10 is capable of identifying areas on the reconstruction, created by ultrasound images, device positions or interpolation of both, associated with cardiac metrics (e.g., voltage, LAT, scar tissue, ablation markers, and the like) selected by the user. The information is associated to sampled or calculated positions in space. The user selection may include more than one cardiac metric type and may be associated with: static display of the reconstruction, dynamic display of the reconstruction, and/or 2D and 3D ultrasound real-time image.

As shown in FIG. 7C, in some embodiments, system 10 displays multiple cardiac chambers. In some embodiments, system 10 is capable of having the same cardiac chambers reconstructed at different times. For example, the left atrium may have an ultrasound-based reconstruction showing fully functional posterior wall motion in the beginning of the procedure and left atrium with reduced posterior wall motion following posterior wall isolation or scar homogenization performed with ablation catheter. In some embodiments displaying multipole cardiac chambers may include display of different views, with switching between views selected by the user. In some embodiments, system 10 may display same static or dynamic reconstruction or 2D and 3D ultrasound images, augmented or colored with different information or visual indications at the same time, in different views, which is discussed in detail further below

As shown in FIG. 7C, in some embodiments, system 10 is configured to project voltage estimates projections 700 of the cardiac structure via 4D renderings, which may be displayed by GUI 401 501, 701. FIG. 7C shows unipolar voltage, however, in some embodiments, system 10 generates bi-polar voltage signals from two EGM electrodes. FIG. 7C illustrates a comparison of the left atrium's voltage landscape before and after a pulmonary vein isolation (PVI) procedure. On the left side, the healthy atrium is depicted, where purple/dark gray coloring indicates areas of high voltage, representing normal electrical conductivity across the atrial tissue. This high-voltage environment is typical in regions of the atrium not affected by arrhythmias.

On the right side, the post-PVI atrium is shown. The red/light gray coloring demarcates the areas of low to no voltage, indicative of successful electrical isolation of the pulmonary veins. The ablation lines creating such isolation are visually represented, effectively segmenting the veins from the rest of the left atrium to prevent arrhythmic signals from entering. The surrounding purple or dark gray areas confirm that the rest of the atrial tissue maintains normal conductivity, signifying that only the targeted regions around the veins have been affected by the ablation. As shown, voltage mapping 700C dynamic reconstruction showcases the efficacy of the PVI procedure in altering the electrical landscape of the atrium to treat conditions like atrial fibrillation. The color-coded/gray scale voltage mapping is advantageous for visualizing the procedure's immediate impact on cardiac electrophysiology.

In some embodiments, system 10 displays any uni-polar or bi-polar in a real-time signal viewer. System 10 may have default signals displayed in various states and under certain conditions and the user has an option to manually arrange, order, color the signals in the signal viewer as well as control the gain and the color of the displayed information.

In some embodiments, system 10 represents the uni-polar and bi-polar voltage information as a color scale and may project such information on GUI 701. As utilized herein color scale or color coded is used interchangeably with gray scale or numeric scaling. For example, some embodiments include projection of the voltage colors on static reconstructions of the cardiac structures. In some embodiments, voltage colors are projected onto a dynamic reconstruction associated with the cardiac cycle (i.e., as cardiac walls are moving with the heart beat the reconstruction is colored according to voltage associated with the specific time along the cardiac cycle. In some embodiments, system 10 projects voltage colors on the 2D and 3D ultrasound image walls. For example, the relevant parts of the anatomy, crossing the ultrasound fan, is colored according to the voltage information, as shown in FIG. 7C.

Referring back now to FIG. 3E in conjunction with FIG. 7D-7E, as mentioned above, system 10 may determine a temporal reference (e.g., signal 310) within the cardiac cycle, or QRS complex, as shown in FIG. 3E. Such temporal reference may be used to generate and display or render local activation time data via LAT map 700D. For clarity, “Local Activation Time” (LAT) in cardiology refers to the time at which a specific area of the heart muscle begins to depolarize during each heartbeat. LAT measurements are advantageous for identifying abnormal cardiac rhythms (i.e., arrhythmias) and their origins within the heart. By mapping LAT across different regions of the heart, as shown in FIG. 7D, physicians may utilize GUI 701 to pinpoint areas of delayed or abnormal electrical activity, which may guide treatment strategies such as catheter ablation for arrhythmias. In some embodiments, LAT data may be generated by reducing the electrode activation time from the reference time for every sampling of the electrode touching the cardiac walls.

For example, in some embodiments, LAT data may be generated by comparing the activation time at each electrode(s) 452 with a reference time (e.g., 710). As catheter(s) 450 comes into contact or engages different parts of the cardiac walls, electrodes on the catheter record the exact time of electrical activation at each point of contact. A reference time is established by using a fiducial point or a consistent phase in the cardiac cycle, such as the onset of the intracardiac complex, discussed above. For sampling points, the time of electrical activation recorded by the electrode is subtracted from the reference time. The result of this subtraction is the Local Activation Time, which represents the relative timing of electrical activation at each point in comparison to the established reference and is displayed via color coded/grayscale rendering in FIG. 7D. Such LAT data is used to create LAT map 700D, which visually represents the sequence of electrical activation across the cardiac tissue. Rendering LAT map 700D in 4D may be advantageous for diagnosing and treating arrhythmias.

In the context of Local Activation Time (LAT) renderings, a fiducial point, shown in FIG. 7E, is a reference point used to measure and compare the timing of electrical activation in different areas of the heart. LAT map 700 C may be implemented to visualize the sequence and timing of electrical activation across the cardiac tissue. As shown in FIG. 7E, fiducial point serves as a baseline or starting point for these measurements, allowing for a standardized comparison of activation times. Doing so is advantageous for identifying areas of abnormal electrical conduction, which may be targets for therapeutic interventions like ablation in conditions such as arrhythmias.

Thus, LAT map 700D is a visual representation of the timing of electrical activation across different regions of heart 32. The color/grayscale bar 712 indicates the relative timing, with red/light gray representing areas of early activation in relation to a central fiducial point within a single heartbeat, while purple/dark gray denotes regions of late activation. This fiducial point serves as a standardized reference for measuring and comparing the electrical activity throughout the cardiac cycle. In this context, the areas of the heart that depolarize first are highlighted in warmer colors (reds and yellows), suggesting these regions are where the electrical impulse initiates. Conversely, cooler colors (greens to purples) or darker gray mark areas with later electrical activation, which indicate delayed conduction. Such maps are instrumental during cardiac procedures, particularly those addressing arrhythmias, as they help identify dysfunctional tissue that may require intervention, like ablation. The precision of LAT maps aids in targeting treatment, ensuring that areas responsible for arrhythmogenic activity are effectively modified to restore normal rhythm.

Accordingly, in some embodiments, system 10 supports projection of Local Activation Time (LAT) information where LAT timing across the cardiac cycle has a color scale 712, which is displayed on GUI 700D. Some embodiments include static LAT colored coded projection on the 2D and 3D ultrasound real-time images (i.e., the chamber wall in the ultrasound image is colored according to the LAT information associated with the static LAT). For example, in some embodiments, the current LAT value for a particular area, represented as a color scale, is projected on the 2D and/or 3D ultrasound image according to the current timestamp within the cardiac cycle. The colors are projected on cardiac walls visible in the ultrasound real-time image. For example, static LAT colored coded projection on static reconstruction and/or static LAT colored coded projection on dynamic reconstruction (i.e., cardiac walls are moving with the heart beat and the LAT map represents a static full cardiac cycle information).

Some embodiments include perpetual dynamic display of the information with a timestamp progressing along the cardiac cycle, for example, emphasizing or indicating the area associated with the current timestamp's LAT value on the dynamic reconstruction. Stated another way, as the timestamp shifts across the cardiac cycle not only the cardiac walls are moving to indicate the heartbeat but also different areas are identified to indicate electrical activation passing through such area.

In some embodiments, system 10 includes real-time cardiac monitoring and alert visualization. As mentioned above, in some embodiments, system 10 may output visual/audio indications to the clinician during cardiac treatment procedures. Such real time monitoring and alert visualization may occur when tiles are in a real-time state (discussed above). For example, via GUI 700, system 10 alerts may provide an advantageously enhance patient safety and procedure efficacy. In such real-time state, tile 704 may be displaying live cardiac imagery configured for instant alert signaling. For example, system 10 may implement a color coding, flashing borders, or overlaid icons to draw attention to the tiles where a potential issue is detected.

For example, analyzing cardiac metrics a may indicate a cardiac anomaly or health concern. For example such cardiac metrics may include: Ejection Fraction (EF) (measures the percentage of blood leaving the heart each time it contracts, crucial for assessing heart function; Cardiac Output (the volume of blood the heart pumps in a minute, indicative of cardiac performance); Myocardial Perfusion (the flow of blood through the heart muscle, important for detecting ischemia); Valve Functionality (assessments of the heart valves' opening and closing, including regurgitation (leakage) and stenosis (narrowing)); Wall Thickness (thickness of the heart muscle, which can indicate hypertrophy or other cardiac conditions; Strain and Strain Rate (measures of the deformation of cardiac tissue during the cardiac cycle, providing insights into myocardial function; Fibrosis Detection (Identification of scar tissue in the heart muscle, which can impact heart function); Blood Flow Velocities (speeds of blood flow through various parts of the heart and vessels, important for assessing hemodynamics); Pressure Gradients (differences in blood pressure across heart valves or vessels, useful for evaluating obstructions or valve issues); Tissue Characterization (analysis of the tissue properties of the heart and vessels, which can help in detecting diseases like cardiomyopathies); Pulmonary Vein Analysis (assessment of the veins returning blood to the heart from the lungs, important in conditions like atrial fibrillation); Left Atrial Volume (an indicator of left ventricular filling pressure and heart failure risk); Aortic Root Dimensions (measurements of the aorta at the level of the root, important for diagnosing conditions like aneurysms); Septal Measurements (thickness and motion of the septum, which can be altered in conditions like hypertrophic cardiomyopathy.

For example, system 10 may be configured for ejection fraction (EF) monitoring. In some embodiments, rendering module 112, in conjunction with the localization module 110, may track the motion (i.e., 4D) of the heart walls to calculate the ejection fraction. In some embodiments, when EF of a patient 12 undergoing a cardiac treatment drops below a predetermined threshold, such EF may indicate possible cardiac dysfunction. Based on detecting EF below the predetermined threshold, GUI may display a visual alert on the corresponding relevant tile.

In some embodiments, system 10 is configured for providing a visual alert of fluid volume in the epicardial space. Because monitoring fluid accumulation is advantageous (e.g., after procedures like pericardiocentesis), system 10 may be configured to determine or detect changes in the fluid volume of an epicardial space. When an abnormal change (increase or decrease) is noted, system 10 may output an audio/visual alert via console 100 and GUI 101, 301, 401, 701.

In some embodiments, system 10 is configured for providing pulmonary vein diameter monitoring. Such monitoring may be advantageous for procedures like pulmonary vein isolation, for preventing stenosis. Thus, system 10 may continuously measure the diameter of the pulmonary veins of patient 12. For example, when a reduction is detected, indicating potential stenosis, system 10 may output an audio/visual alert via console 100, 400 and/or GUI 101, 301, 401, 701, via the affected tile (e.g., 704h), and may display an audio/visual alert.

In some embodiments, system 10 may trigger an audio/visual alert based on a position or movement of a catheter (e.g., 150, 250). For example, when catheter 250 moves out of a desired location within the cardiac anatomy, possibly due to patient movement or catheter manipulation, localization module 108 may cause rendering module 112 to output a visual and/or audio alert via console 100, GUI 501, 701. Implementing such visual/audio alerts ensures catheter 150, 250 remains in the correct position for effective treatment.

In some embodiments, system 10 may trigger an audio/visual alert based on cardiac wall stretch, or so-called “Tenting”. For example, during procedures like mitral valve repair, excessive stretching of the cardiac wall 58 may be detrimental. Thus, system 10 may measure the degree of wall movement or deformation, and provide a visual/audio alert to the clinician when the stretching exceeds safe limits.

Thus, the foregoing alert framework of system 10 may be configured to not only notify but also guide the clinician to the appropriate response. For example, by providing a visual alert that recommends adjusting the catheter position, managing fluid volume, or taking other corrective measures. The integration of these alerts into GUI 501, 701 ensures that the clinician is informed in real-time, allowing for immediate action to be taken, which is essential in the dynamic environment of cardiac procedures.

In some embodiments, predictive module 116 and/or resources 120 may include predictive model algorithms which may be trained to recognize and identify physiological features such as lesions, scars, septal defects, patent foramen ovale, wall motion and ejection fraction, valve leaks and stenosis, thrombus, myxoma, tissue thickness, optimized device placement for CRT/left ventricular assist devices. The predictive model algorithms incorporate ultrasound imaging with intra-cardiac voltage and catheter localization. Predictive model algorithms may be trained to identify fiducial points that would maximize the quality of ultrasound-mased image.

In some embodiments, external resources 120 may include a predictive model training program. Some embodiments include training predictive model algorithms to recognize and identify various physiological features. Such training requires a systematic approach that involves data collection, preprocessing, model selection, training, and validation.

The embodiment herein provides a predictive model capable of accurately identifying features such as lesions, septal defects, patent foramen ovale, wall motion and ejection fraction, valve leaks and stenosis, thrombus, myxoma, tissue thickness, and optimized device placement for Cardiac Resynchronization Therapy (CRT) and left ventricular assist devices. The integration of ultrasound imaging with intra-cardiac voltage and catheter localization is advantageous in such process predictive model training.

In some embodiments, external resources 120 may include a comprehensive dataset comprising ultrasound images and corresponding intra-cardiac voltage data. Such dataset advantageously includes a wide range of cases, covering various conditions such as lesions, valve leaks, and other cardiac features. Such data should also represent different patient demographics to ensure the model's generalizability.

External resources 120 may include data annotation, which employs expert cardiologists to annotate the images, marking the regions of interest like lesions, thrombus, and valve abnormalities. Such annotations will serve as the ground truth for training the AI models.

In some embodiments, external resources may facilitate processing the image data to enhance image quality and extract relevant features. Such enhancement techniques like noise reduction, contrast enhancement, and edge detection may be applied to ultrasound images. In some embodiments, such intra-cardiac voltage data may be normalized to ensure consistency across different measurements.

In some embodiments, various machine learning models may be implemented. One embodiment includes use of a convolutional neural networks (CNNs), which are advantageous for image recognition tasks. In some embodiments, for integrating intra-cardiac voltage data, various hybrid machine learning models that can process both image and numerical data may be implemented.

In some embodiments, feature extraction algorithms may be employed for extracting features from the ultrasound images and voltage data. For example, by edge detection techniques to identify septal defects or employ texture analysis to detect tissue thickness variations. In some embodiment training the AI model includes training dataset by using the annotated and preprocessed data to train the AI model. Advantageously, such training set is diverse and representative of different cardiac conditions.

Some embodiments include incorporating catheter localization data for training the model. This step is advantageous for tasks like optimized device placement, where precise positioning is advantageous. In some embodiments, training the model includes hyper parameter tuning. For example, some embodiments optimize the model by adjusting hyper parameters like learning rate, batch size, and the number of layers in the neural network.

Some embodiments may employ cross-validation techniques to evaluate the model's performance. This involves dividing the dataset into different subsets and using each subset for testing and validation. Some embodiments implement use of metrics like accuracy, precision, recall, and F1-score to assess the model's ability to identify physiological features correctly. In other embodiments, iterative improvement based on the validation results may iteratively improve the model. Such improvement may involve re-tuning hyper parameters, augmenting the training dataset, or modifying the feature extraction process.

In some embodiments, predictive module 116 is configured for providing increased image quality via Fiducial Point Training (FPT). In FPT, a predictive model is specifically trained to identify fiducial points in the ultrasound images that maximize image quality. This involves recognizing key anatomical landmarks and ensuring the clarity for such landmarks in the ultrasound imaging. Additional optimization techniques may include adjusting imaging parameters based on identified fiducial points, thereby enhancing the quality of the ultrasound-based image.

Accordingly some embodiments employed herein provide for training an AI model for predictive analysis in cardiac imaging in a multifaceted process that requires careful data collection and preprocessing, judicious model selection, rigorous training, and thorough validation as described above. By continuously refining the model based on feedback and performance metrics, the AI system can become a powerful tool in cardiac diagnostics and treatment planning.

Thus, the embodiments described herein aim to revolutionize the field of cardiac imaging, providing an integrated, multi-dimensional view of cardiac anatomy and function. Its innovative approach significantly enhances diagnostic and therapeutic capabilities in the realm of interventional cardiology, promising a new era of precision and clarity in cardiac care. In contrast to the conventional single-ended data viewpoints of conventional imaging devices, which offers a limited perspective, the embodiments described above employ multi-transducer technology to provide a comprehensive, multi-level data analysis. Such advantageous approaches mark a significant leap from conventional methods, delivering high-resolution, dynamic insights into complex anatomical and functional details with unparalleled precision and technological sophistication.

The embodiments described herein may be embodied in systems, apparatus, methods, computer programs and/or articles depending on the desired configuration. Any methods or the logic flows depicted in the accompanying figures and/or described herein do not necessarily require the particular order shown, or sequential order, to achieve desirable results. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. The implementations described above can be directed to various combinations and subcombinations of the disclosed features and/or combinations and subcombinations of further features noted above. Furthermore, above described advantages are not intended to limit the application of any issued claims to processes and structures accomplishing any or all of the advantages.

Furthermore, any reference to this disclosure in general or use of the word “embodiment” in the singular is not intended to imply any limitation on the scope of the claims set forth below. Multiple embodiments may be set forth according to the limitations of the multiple claims issuing from this disclosure, and such claims accordingly define the embodiment(s) herein, and their equivalents, that are protected thereby.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

1. A system (10) for surgical imaging utilizing imaging devices configured to be inserted into a body of a patient (12), the system comprising:
a display device (101);
a first catheter (150) having a first transducer, the first transducer configured to sense an anatomical structure (32) of the body of the patient and output a first set of imaging data;
a second catheter (150B) having a second transducer configured to sense the anatomical structure and output a second set of imaging data;
a processor (104) in communication with a memory (106), the memory storing executable instructions that when executed by the processor configure the system for:
receiving, simultaneously, the first set of imaging data and the second set of imaging data;
determining, a position (606) of the anatomical structure, based on the first set of imaging data and the second set of imaging data;
displaying, on the display device, a real-time rendering (700) of the anatomical structure based on the position of the anatomical structure;

2. The system of claim 1, wherein displaying the real-time rendering includes displaying a first anatomical structure on a first portion of the display device, and displaying a second anatomical structure on a second portion of the display device.

3. The system of claim 2, wherein the first anatomical structure corresponds to the first set of imaging data, and wherein the second anatomical structure corresponds to the second set of imaging data.

4. The system of claim 1, further comprising,
a first plurality of electrodes positioned on a first catheter shaft of the first catheter; and
a second plurality of electrodes positioned on a second catheter shaft of the second catheter,
wherein the first and second plurality of electrodes are configured to output impedance data.

5. The system of claim 4, wherein, when executed by the processor, the executable instructions further configure the system for:
determining a local position and an orientation of an individual electrode of the first or second plurality of electrodes based on the impedance data and/or a proximity of the individual electrode to the first transducer and/or the second transducer; and
adjusting the real-time rendering based on the local position and the orientation of the individual electrode;
displaying, on the display device, an overlay of the real-time rendering, the overlay including spatial data corresponding to the local position of the individual electrode.

6. The system of claim 1, wherein the second catheter includes a third transducer spaced apart from second transducer, the second transducer being positioned in a first orientation, the third transducer being positioned in a second orientation, wherein the first orientation and the second orientation are different.

7. The system of claim 6, wherein the first orientation and the second orientation differ by at least 30 degrees of rotation from a central axis of the second transducer or the third transducer, and wherein the second transducer and the third transducer are spaced apart by at least 10 cm.

8. The system of claim 1, wherein the first catheter further includes:
a handle coupled to a catheter shaft; and
a shape sensing fiber optic coupled to an interior wall of the catheter shaft, the shape-sensing fiber optic wire configured to determine a position and orientation of the first plurality of electrodes and/or the first transducer.

9. The system of claim 1, wherein the executable instructions further configure the system for:
receiving streaming imaging data and timestamping individual cardiac cycles
determining, based on the imaging data, an occurrence corresponding to a slip of the catheter, the patient moving, or both; and
displaying an alert based the occurrence.

10. The system of claim 1, wherein the executable instructions further configure the system for:
receiving, a user selection of a first cardiac metric and a second cardiac metric;
displaying, in response to the user selection, a static or dynamic reconstruction of the heart corresponding to the first cardiac metric on a first portion of the display device; and
displaying, in response to the user selection of a second cardiac metric, a second static or dynamic reconstruction of the heart corresponding to the second cardiac metric on a second portion of the display device.

11. An apparatus (350) configured to be surgically positioned into a body of a patient (12), the apparatus comprising:
a catheter shaft (357) coupled to a handle (353);
a plurality of electrodes (352) positioned on the catheter shaft, the plurality of electrodes including a magnetic sensor (372); and
a transducer (354) configured to sense one or more anatomical structures of the body of the patient and output a first set of imaging data, wherein the magnetic sensor tracks a location and position of the plurality of electrodes and/or the transducer.

12. The apparatus of claim 11, wherein the handle is rotatably actionable and configured to cause at least 180 degrees of deflection in the catheter shaft.

13. The apparatus of claim 11, wherein the plurality of electrodes includes between 4-20 electrodes configured for voltage sampling.

14. The apparatus of claim 11, wherein the plurality of electrodes includes a reference electrode positioned on the catheter shaft and spaced apart from the handle by substantially at least 4 cm.

15. The apparatus of claim 14, wherein the reference electrode outputs a reference signal configured for grounding or a baseline measurement point.

16. The apparatus of claim 11, wherein the catheter shaft further comprises a shape sensing fiber optic coupled to an interior wall of the catheter shaft, the shape-sensing fiber optic wire configured to determine the position and orientation of at least one of: an individual one of the plurality of electrodes, the first transducer, or the magnetic sensor.

17. The apparatus of claim 11, wherein the catheter shaft further includes one or more splines coupled to the catheter shaft, wherein the plurality of electrodes and/or the transducer are positioned along the one or more splines, and wherein the transducer is coupled to a magnetic sensor, the magnetic sensor configured for tracking six degrees-of-freedom (6DOF).

18. A method for surgical imaging of anatomical structures of a body of a patient utilizing a system for surgical imaging including a display device and one or more catheters, the method comprising:
establishing connections to a first transducer, a second transducer, and a plurality of electrodes;
receiving, from the first transducer, a first data stream corresponding to an anatomical structure of the body of the patient;
receiving, from the second transducer, a second data stream corresponding to the anatomical structure;
receiving, from the plurality of electrodes, voltage data corresponding to impedance values;
determining, based on the first data stream, the second data stream, and the impedance values, a position and orientation of the anatomical structure, the plurality of electrodes, and the first transducer, and/or the second transducer;
reconstructing, based on the position and orientation of the anatomical structure, a single real-time imaging stream of the anatomical structure; and
transmitting the single-real time imaging stream for display on a display device.

19. The method of claim 18, wherein the displaying the dynamic anatomical reconstruction includes:
determining voltage estimates based on the voltage data;
overlaying the voltage estimates on the real-time stream;
receiving, a user selection of a cardiac metric;
displaying, in response to a first user selection of a first cardiac metric, a static or dynamic reconstruction of the heart corresponding to the first cardiac metric; and
displaying, in response to a second user selection of a second cardiac metric, a second static or dynamic reconstruction of the heart corresponding to the second cardiac metric.

20. The method of claim 18, wherein displaying the first or second cardiac metric includes displaying an alert when the first cardiac metric or the second cardiac metric exceeds or falls below a threshold value.
